(19) United States
(12) Patent Application Publication
(10) Pub. No.: US 2022/0220430 A1
(43) Pub. Date: Jul. 14, 2022

(54) METHODS OF MONITORING CELL CULTURE MEDIA

(71) Applicant: Bristol-Myers Squibb Company, Princeton, NJ (US)

(72) Inventors: Pegah ABADIAN, Devens, MA (US); Kathryn ARON, Devens, MA (US)

(73) Assignee: Bristol-Myers Squibb Company, Princeton, NJ (US)

(21) Appl. No.: 17/613,904

(22) PCT Filed: May 22, 2020
(86) PCT No.: PCT/US2020/034403

§ 371 Date: Nov. 23, 2021

Related U.S. Application Data
(60) Provisional application No. 62/852,230, filed on May 23, 2019.

(51) Int. Cl. C12M 1/34; G01N 21/65; G01P 13/00

(52) U.S. Cl. CPC C12M 41/32; C12N 5/0018; G01P 13/00; G01N 21/65

(57) ABSTRACT

This disclosure provides a method of fingerprinting and analyzing cell culture samples using Raman spectroscopy to detect cell culture media preparation errors, degradation, or other changes in the media that may render it suboptimal for cell culture use.

### CROSS-REFERENCE TO RELATED APPLICATIONS

This application claims the priority benefit of U.S. Provisional Application No. 62/852,230, filed May 23, 2019 and is hereby incorporated by reference in its entirety.

### BACKGROUND OF THE DISCLOSURE

Sub-optimal media utilized for upstream bioprocessing can cost hundreds of thousands of dollars in terms of lost labor and delayed production. Media is deemed sub-optimal when prepared incorrectly, i.e. when a particular component is accidentally excluded or added at the wrong amount or when utilized past its optimal expiry. Media quality is an important performance parameter that is typically evaluated by HPLC functional testing, such as by amino acid analysis, in a 14-day production bioreactor process with daily sampling and feeding, which is time and resource-intensive. Use of sub-optimal media can result in reduced titers and/or altered product attributes. It is important, however, to have analytical methods that can properly measure media composition to ensure the physical presence of critical components, and to monitor expiry via measurement of important markers of degradation. Therefore, there is a need to develop cost effective and accurate methods that can measure the condition of culture media.

### SUMMARY OF THE DISCLOSURE

The present disclosure is related to a method of monitoring changes in cell culture media that could affect its effectiveness in growing cells in any setting. One aspect of the present disclosure is directed to the measurement of a Raman spectrum of a cell culture media sample to compare it to known spectra of the components of the cell culture media. The methods of the present disclosure are directed to a method for fingerprinting a cell culture media and/or identifying optimal storage conditions for a cell culture media using Raman spectroscopy, comprising: collecting a Raman spectrum of the cell culture media, the cell culture media containing a mixture of one or more components, wherein the Raman spectrum of the cell culture media is compared to one or more reference spectra associated with each of the one or more components or one or more reference spectra associated with a reference media composition. The methods of the present disclosure are also directed to a method for identifying the rate of change of particular components in cell culture media and/or monitoring the changes of a cell culture media in real-time using Raman spectroscopy, comprising: collecting a Raman spectrum of the cell culture media (“collected spectrum”), wherein the Raman spectrum of the cell culture media is compared to a reference spectrum associated with each of the one or more components.

In some aspects, the reference spectrum is to be free of degradation. In some aspects, the collecting of a Raman spectrum of the cell culture media is conducted at least about every hour, at least about every two hours, at least about every three hours, at least about every four hours, at least about every five hours, at least every six hours, at least every seven hours, at least about every eight hours, at least about every nine hours, at least about every ten hours, at least about every 11 hours, at least about every 12 hours, at least about every 13 hours, at least about every 14 hours, at least about every 15 hours, at least about every 16 hours, at least about every 17 hours, at least about every 18 hours, at least about every 19 hours, at least about every 20 hours, at least about every 21 hours, at least about every 22 hours, at least about every 23 hours, or at least about every 24 hours.

In some aspects, the Raman spectrum has an average of at least 5 data points, at least 10 data points, at least 15 data points, at least 16 data points, at least 17 data points, at least 18 data points, at least 19 data points, at least 20 data points, at least 21 data points, at least 22 data points, at least 23 data points, at least 24 data points, at least 25 data points, at least 26 data points, at least 27 data points, at least 28 data points, at least 29 data points, at least 30 data points, at least 31 data points, at least 32 data points, at least 33 data points, at least 34 data points, at least 35 data points, at least 36 data points, at least 37 data points, at least 38 data points, at least 39 data points, at least 40 data points, at least 41 data points, at least 42 data points, at least 43 data points, at least 44 data points, at least 45 data points, at least 46 data points, at least 47 data points, at least 48 data points, at least 49 data points, or at least 50 data points. In some aspects, each data point is measured every 10 seconds, every 15 seconds, every 20 seconds, every 25 seconds, every 30 seconds, every 35 seconds, every 40 seconds, every 45 seconds, every 50 seconds, every 55 seconds, or every 60 seconds.

The methods of the present disclosure also involve analysis of the raw Raman spectra collected from the samples. In some aspects, the Raman spectrum is analyzed by a multivariate analysis. In some aspects, the multivariate analysis is a principle component analysis (PCA). In some aspects, the PCA generates a PC score for the Raman spectrum. In some aspects, the multivariate analysis is a partial least squares analysis (PLS) and wherein the analysis produces a calibration prediction model. In some aspects, the method further comprises comparing the collected spectrum of the cell culture media. In some aspects, the comparing comprises a comparison of a PC score of the collected spectrum to a reference PC score of the reference spectrum.

In some aspects, the method further comprises determining that the cell culture media is degraded when the PC score of the collected spectrum is different from the reference PC score of the reference spectrum at least by about 10, at least by about 11, at least by about 12, at least by about 13, at least by about 14, at least by about 15, at least by about 16, at least by about 17, at least by about 18, at least by about 19, at least by about 20, at least by about 21, at least by about 22, at least by about 23, at least by about 24, at least by about 25, at least by about 26, at least by about 27, at least by about 28, at least by about 29, or at least by about 30. In some aspects, the method further comprises determining that the cell culture media is degraded when the PC score of the collected spectrum is higher than the reference PC score of the reference spectrum. In some aspects, the method further comprises determining that the cell culture media is degraded when the PC score of the collected spectrum is lower than the reference PC score of the reference spectrum.

In some aspects, the cell culture media is degraded and said degraded cell culture media reduces the viability of the cells in culture as compared to a non-degraded media by about 1%, by about 2%, by about 3%, by about 4%, by about 5%, by about 6%, by about 7%, by about 8%, by about 9%, or by about 10%.

In some aspects, the cell culture media is degraded and said degraded cell culture media reduces the viable cell density of the cells in culture as compared to a non-degraded media by about 1×10⁶ cells/mL, by about 2×10⁶ cells/mL, by about 3×10⁶ cells/mL, by about 4×10⁶ cells/mL, by about 5×10⁶ cells/mL, by about 6×10⁶ cells/mL, by about 7×10⁶ cells/mL, by about 8×10⁶ cells/mL, by about 9×10⁶ cells/mL, by about 10×10⁶ cells/mL, by about 11×10⁶ cells/mL, or by about 12×10⁶ cells/mL.

In some aspects, the cell culture media is degraded and said degraded cell culture media reduces an antibody production titer of the cells in culture as compared to a non-degraded media by about 0.1 g/L, by about 0.2 g/L, by about 0.3 g/L, by about 0.4 g/L, by about 0.5 g/L, by about 0.6 g/L, by about 0.7 g/L, by about 0.8 g/L, by about 0.9 g/L, by about 1.0 g/L, by about 1.1 g/L, by about 1.2 g/L, by about 1.3 g/L, by about 1.4 g/L, by about 1.5 g/L, by about 1.6 g/L, by about 1.7 g/L, by about 1.8 g/L, by about 1.9 g/L, by about 2.0 g/L, by about 2.1 g/L, by about 2.2 g/L, by about 2.3 g/L, by about 2.4 g/L, by about 2.5 g/L, by about 2.6 g/L, by about 2.7 g/L, by about 2.8 g/L, by about 2.9 g/L, or by about 3.0 g/L.

In some aspects, a marker is added to the cell culture media. In some aspects, the marker is selected from the group consisting of lysine (Lys), histidine (His), asparagine (Asn) and arginine (Arg). In some aspects, the marker is tyrosine. In some aspects, the marker is not glucose. In some aspects, the marker is not lactate. In some aspects, the marker is selected from the group consisting of cyanocobalamin (B12), folic acid (B9), niacinamide (B3), pyridoxal HCl (B6(AL)), and pyridoxine HCl (B6(INE)).

In some aspects, the Raman spectrum is measured in the range of from about 500 cm⁻¹ to about 1700 cm′, from about 500 cm⁻¹ to about 1800 cm′, from about 500 cm⁻¹ to about 1900 cm⁻¹, from about 500 cm⁻¹ to about 2000 cm⁻¹, from about 500 cm⁻¹ to about 2100 cm⁻¹, from about 500 cm⁻¹ to about 2200 cm⁻¹, from about 500 cm⁻¹ to about 2300 cm⁻¹, from about 500 cm⁻¹ to about 2400 cm⁻¹, from about 500 cm⁻¹ to about 2500 cm⁻¹, from about 500 cm⁻¹ to about 2600 cm⁻¹, from about 500 cm⁻¹ to about 2700 cm⁻¹, from about 500 cm⁻¹ to about 2800 cm⁻¹, from about 500 cm⁻¹ to about 2900 cm⁻¹, or from about 500 cm⁻¹ to about 3000 cm⁻¹. In some aspects, the Raman spectrum is measured in the range of 500 cm⁻¹ to 3000 cm⁻¹.

In some aspects, the method further comprises determining that the cell culture media is stable when the PC score of the collected spectrum is the same as or similar to the reference PC score of the reference spectrum by about 10 or less, by about 9 or less, by about 8 or less, by about 7 or less, by about 6 or less, by about 5 or less, by about 4 or less, by about 3 or less, by about 2 or less, or by about 1 or less.

In some aspects, the cell culture media is determined for storage for about eight days, for about nine days, for about ten days, for about 11 days, for about 12 days, about 15 day, for about 16 days, for about 17 days, for about 18 days, for about 19 days, for about 20 days, for about 21 days, for about 22 days, for about 23 days, for about 24 days, for about 25 days, for about 26 days, for about 27 days, for about 28 days, for about 29 days, for about 30 days, for about a month, for about 1.5 months, for about 40 days, for about 50 days, for about 60 days, for about two months, for about 70 days, for about 80 days, for about 90 days, for about three months, for about 100 days, for about 110 days, for about 120 days, for about 4 months, for about five months, or for about six months.

The present disclosure is also related to an apparatus for carrying out measurements of Raman spectra using an apparatus capable of analyzing cell culture media. In some aspects, the apparatus comprises a mixture of one or more components in the cell culture media, the apparatus comprising: a cell culture media sample holder for holding the cell culture media sample; a laser source for illuminating the cell culture media sample held by the cell culture media sample holder; a particle motion detector positioned to detect motion of one or more components in the cell culture media in the cell culture media sample held by the cell culture media sample holder; and a spectral detector positioned to receive a spectrum from the cell culture media sample resulting from illumination by the laser source.

### BRIEF DESCRIPTION OF THE DRAWINGS

FIG. 1 shows an energy diagram of transitions between the vibrational energy levels corresponding to the processes of infrared (IR) absorption, Rayleigh scattering and Raman scattering (Stokes and anti-Stokes). E0 and E1 (E0+h vm) are the electronic ground and excited states. v0 and vm are the vibrational ground and excited states.

FIGS. 2A, 2B, and 2C. FIG. 2A shows a schematic of the T-connector, connected to the feed media bag and run through Raman using the peristaltic pump, and FIG. 2B shows a picture of the T-connector set-up and the attached tubing. FIG. 2C shows glass vials and an amber beaker used for offline measurements. Glass vials were covered with aluminum foil and the amber beaker was covered with black polypropylene to eliminate light interference. The T-connector is a 316L stainless steel pilot scale flow cell with ¾ inch sanitary flange inlet/outlet for a 12 mm OD probe.

FIG. 3 shows the PC1 score plot against the number of measurement of the real-time feed media analysis. The measurements were done every 3 hours using Raman spectroscopy. For example, on the X axis, a measurement of 5 indicates a measurement 15 hours from the initial monitoring. The arrow represents the measurement where the Raman spectra began to change, at approximately 90 hours after initial monitoring.

FIGS. 4A and 4B. FIG. 4A shows the PC1 loading against the Raman shift of the real-time feed media analysis representing the wavenumbers at which the major changes in the media occurred. FIG. 4B shows the Raman spectra of tyrosine crystals. (Freire, P., et al., Raman Spectroscopy of Amino Acid Crystals. Raman Spectroscopy and Applications, 2017).

FIG. 5 shows a PC1 score plot of different phosphate levels added to B9 Basal media. The first points are showing the phosphate level of 1.5 g/L and the last points represent the samples with 3 g/L phosphate.

FIG. 6 shows a PC1-PC2 plane score plot of the feed/basal media forced degradation.

FIG. 7 shows the PC2 score plot representing the changes detected in the basal media by Raman testing due to heat (H) or light (L) forced degradation as well as normal aging at 4° C. The time of storage is indicated in weeks (e.g., 1W) or months (3M). Note that the 3M time point is not represented at scale.

FIGS. 8A, 8B, and 8C show the production VCD (FIG. 8A), viability (FIG. 8B), and antibody titer (FIG. 8C) when differently aged basal and fresh feed were used.

FIG. 9 shows the changes in the amino acids levels of basal media due to light (L) and heat (H) degradation. The dashed lines show the trend of the changes due to light degradation and the solid lines represent the changes due to heat degradation.

FIG. 10 shows the PC1 score plot of feed media due to light (L) and heat (H) degradation over time (W for weeks or M for months).

FIGS. 11A to 11C shows that differently aged feed media and fresh basal can affect the production results. FIG. 11A shows the viable cell density (VCD). FIG. 11B shows the viability. FIG. 11C shows the antibody titer (C) results.

FIG. 12A shows the calibration curve for the prediction model of arginine. FIG. 12B shows the first latent variant of the spiking experiment that was used to build a model. FIG. 12C shows the Raman spectra of arginine solution in water. The dashed lines confirm the major variation in LV1 plot was related to Raman shifts of arginine.

FIGS. 13A-13H show the prediction models for concentration of 4 amino acids and 4 vitamins. The fit line is the fitted line of the calibration curve, which was built using the known concentrations for each chemical, shown by circles. The diamond shows the predicted level of an unknown level of Arg (FIG. 13A), Asn (FIG. 13B), His (FIG. 13C), Lys (FIG. 13D), B6(AL) (FIG. 13E), B6(INE) (FIG. 13F), B9 (FIG. 13G), or B12 (FIG. 13H). The 1:1 line shows a 1:1 ratio between measured and predicted values, measured values are final spiked concentrations. The stronger the prediction model the better fit and 1:1 line overlay.

FIGS. 14A-14H show the % recoveries at each concentration level for each chemical. The circles represent the points used to build the calibration curve and the diamond is the predicted concentration using the calibration curve with respect to Arg (FIG. 14A), Asn (FIG. 14B), Lys (FIG. 14C), His (FIG. 14D), B6(AL) (FIG. 14E), B6(INE) (FIG. 14F), B9 (FIG. 14G), B12 (FIG. 14H).

FIG. 15A shows reference spectra for amino acids arginine, asparagine, histidine and lysine. FIGS. 15B and 15C show reference spectra for vitamins cyanocobalamin (B12), folic acid (B9), niacinamide (B3), pyridoxal HCl (B6(AL)), and pyridoxine HCl (B6(INE)).

### DETAILED DESCRIPTION OF THE DISCLOSURE

The present disclosure is directed to a method that can monitor real-time changes in media composition, identify specific errors in media preparation, and monitor media stability. Raman spectroscopy, an example of a label-free fingerprinting technique, can be utilized for this purpose. The present disclosure also shows that Raman based models can be built to quantitatively model the amino acids and vitamin components in the media. The present disclosure shows that Raman technique can be utilized as an analytical testing to replace functional testing and minimize risk from lost batches.

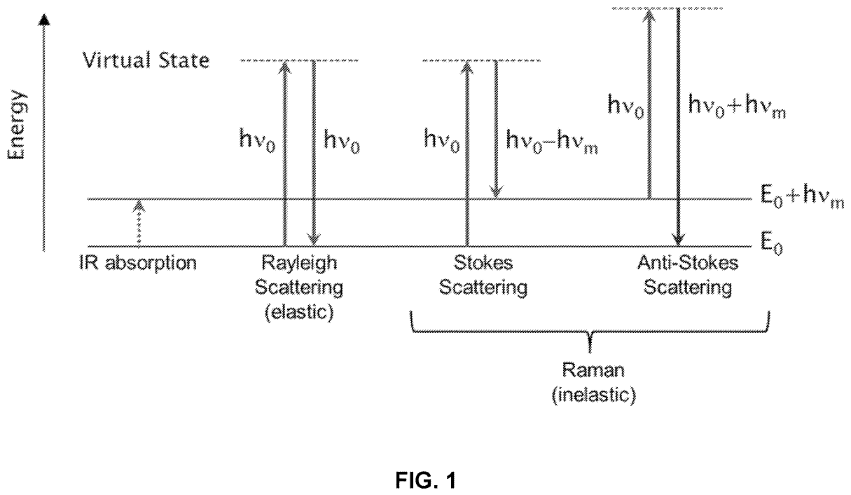

Raman is a spectroscopy technique based on inelastic scattering of monochromatic light, usually from a laser source, where the frequency of the photons of light shifts upon interaction with a chemical bond. This shift, called a “Raman shift”, provides useful information about the rotational, vibrational and other low frequency transitions of that bond. Since every molecule has a unique set of chemical bonds that constitutes its structure, Raman can be utilized as a “fingerprinting technique” that forms signature patterns unique to each molecule. FIG. 1 illustrates the principles of Raman scattering and compares it to two alternative spectroscopy techniques (IR and Raleigh).

Some of the advantages of Raman include its ability to footprint molecules without the need for sample labels or sample preparation. This provides the means to measure in real-time, which makes it a useful technique for Process Analytical Technology (PAT) applications.

### I. Terms

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

As used herein, the term “Raman scattering” refers to a spectroscopic technique used to observe vibrational, rotational, and other low-frequency modes in a system. It relies on inelastic scattering, or Raman scattering, of monochromatic light, usually from a laser in the visible, near infrared, or near ultraviolet range. The laser light interacts with molecular vibrations, phonons or other excitations in the system, resulting in the energy of the laser photons being shifted up or down. The shift in energy gives information about the vibrational modes in the system. A variety of optical processes, both linear and nonlinear in light intensity dependence, are fundamentally related to Raman scattering. As used herein, the term “Raman scattering” includes, but is not limited to, “stimulated Raman scattering” (SRS), “spontaneous Raman scattering”, “coherent anti-Stokes Raman scattering” (CARS), “surface-enhanced Raman scattering” (SERS), “Tip-enhanced Raman scattering” (TERS) or “vibrational photoacoustic tomography”.

The term “degradation” and/or “degraded”, as used herein, refers to the reduction in a composition's ability to be used effectively as intended. For example, cell culture media may be degraded by exposure to heat, light, humidity, or other environmental conditions that can cause unwanted effects of the components of the media. Changes in concentration over time of various components of the cell culture may also result in degraded cell culture media. Errors in preparation of media may also be detected as degraded cell culture media using the techniques of the present disclosure. In all instances, degraded cell culture media may not be as effective at maintaining the viability and/or growth of cell culture as compared to media that is not degraded. Degraded cell culture may be less effective at upstream biomanufacturing as compared to non-degraded media, as it may affect the upstream cell growth and/or protein expression rates, cell count and/or cell densities, or total cell viability during upstream manufacturing.

The terms “culture”, “cell culture” and “eukaryotic cell culture” as used herein refer to a cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the cell population and the medium in which the population is suspended.

The terms “media”, “medium”, “cell culture medium”, “culture medium”, “tissue culture medium”, “tissue culture media”, and “growth medium” as used herein refer to a solution containing nutrients which can be used to nourish growing cultured host cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant glycoproteins or proteins, for example, but not limited to, hormones, cytokines, interleukins and other signaling molecules.

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

The term “upstream process,” “upstream cell culture process” or “upstream manufacturing process”, as used herein, generally refers to the first step or steps in a manufacturing process where microbes or cells are grown, e.g. bacterial or mammalian cells, in vessels such as bioreactors. Upstream processing involves all the steps related to inoculum development, media development, protein expression, improvement of inoculum by genetic engineering processing, and optimization of growth kinetics.

The term “batch culture” or “batch reactor process” as used herein refers to a method of culturing cells in which all the components that will ultimately be used in culturing the cells, including the medium (see definition of “medium” below) as well as the cells themselves, are provided at the beginning of the culturing process. A batch culture is typically stopped at some point and the cells and/or components in the medium are harvested and optionally purified.

The term “fed-batch culture” or “fed-batch reactor process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

As used herein “perfusion” or “perfusion culture” or “perfusion reactor process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system. By “non-anchorage dependent cells” is meant cells propagating freely in suspension throughout the bulk of a culture, as opposed to being attached or fixed to a solid substrate during propagation. The continuous cell culture system can comprise a cell retention device similar to that used in a perfusion system, but that allows continuous removal of a significant portion of the cells, such that a smaller percentage of the cells are retained than in perfusion culture. By “cell retention device” is meant any structure capable of retaining cells, particularly non-anchorage dependent cells, in a particular location during cell culture. Non-limiting examples include microcarriers, fine mesh spin filters, hollow fibers, flat plate membrane filters, settling tubes, ultrasonic cell retention devices, and the like, that can retain non-anchorage dependent cells within bioreactors. Polypeptides and/or viruses of interest (e.g., a recombinant polypeptide and/or recombinant virus) can be recovered from the cell culture system, e.g., from medium removed from the cell culture system.

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. A heavy chain may have the C-terminal lysine or not. The term “antibody” can include a bispecific antibody or a multispecific antibody.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody may consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The terms “polypeptide” or “protein” or “product” or “product protein” or “amino acid residue sequence” are used interchangeably. The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

### II. Methods of Monitoring Media

The present disclosure provides a highly effective approach to analyze the degradation of cell culture media using Raman Spectroscopy by comparing a Raman spectrum obtained from a test cell culture media and comparing it to the Raman spectrum of one or more known components of the cell culture media that are not degraded. The respective Raman spectra can be acquired from the media itself or one or more components thereof. For example, one or a plurality of Raman spectra can be acquired on a single component of the cell culture media, such as tyrosine or other amino acids. Alternatively or additionally, one or a plurality of Raman spectra can be acquired from a cell culture media sample to be tested for degradation. In this manner, precipitates or other elements of degradation can be detected in a cell culture media preparation.

The methods of the present disclosure comprise controlling a Raman spectrometer to collect a Raman spectrum of a targeted volume within the sample so as to collect a Raman spectrum of a target cell culture media. The method further comprises obtaining reference spectra uniquely associated with the known components of the cell culture media. The reference spectra comprise at least one spectral measurement of one or more cell culture media components. Moreover, the method also comprises comparing, using a processing device, the reference spectra to the collected spectrum, and identifying whether there is at least one unwanted molecular composition within the collected spectrum based upon the comparison of the reference spectra to the collected spectrum. In this regard, the method yet further comprises providing an indication as to whether cell culture media degradation has occurred based on analysis of the collected Raman spectrum where at least one unwanted molecular composition is identified within the collected spectrum, and stopping the manufacturing process and/or discarding the degraded media prior to use during a manufacturing process where degradation is detected in the collected Raman spectrum. Unwanted molecular compositions can be identified by identifying unwanted molecules or precipitates based upon an analysis of a difference spectrum computed between the collected spectrum and the reference spectrum of a cell culture media composition that is free of degradation.

According to further aspects of the present disclosure, a system that detects degradation in a manufacturing process comprises an optical imaging system and a processor. The optical imaging system implements a Raman spectrometer that is controlled to direct a laser to a targeted volume within a sample area so as to collect a Raman spectrum of cell culture media. The processor is coupled to the optical imaging system. In this manner, the processor executes program code to receive the Raman spectrum, and to access reference spectra that describes the known components. The reference spectra comprise spectral measurements of one or more cell culture media components. The processor further executes program code to compare the reference spectra to the collected spectrum, and identify whether there is at least one unwanted molecular composition within the collected spectrum based upon the comparison of the reference spectra to the collected spectrum. In addition, the processor executes program code to provide an indication as to whether degradation is present in the collected Raman spectrum based upon whether at least one unwanted molecular composition is identified within the collected spectrum, and stop the manufacturing process and/or discard the degraded media prior to use during a manufacturing process where degradation is detected in the collected Raman spectrum. The computing unit can be configured to determine the reaction of the biological object to the at least one substance by means of a statistical evaluation of the Raman spectrum acquired before administration of the at least one substance and the Raman spectrum acquired after administration of the at least one substance. The statistical evaluation can comprise a principal component analysis (PCA), a partial least squares analysis (PLS), a cluster analysis and/or a linear discriminant analysis (LDA). In some aspects, the statistical evaluation can comprises a PCA.

The methods of the present disclosure are also useful for monitoring markers added to cell culture media with known Raman spectra. In some aspects, the added markers are one or more amino acids. In some aspects, the added markers are one or more amino acids selected from the group of lysine (Lys), histidine (His), asparagine (Asn), arginine (Arg), alanine (Ala), aspartic acid (Asp), cysteine (Cys), glutamic acid (Glu), glutamine (Gln), glycine (Gly), isoleucine (Ile), leucine (Leu), methionine (Met), phenylalanine (Phe), proline (Pro), serine (Ser), Threonine (Thr), Tryptophan (Trp), tyrosine (Tyr), and valine (Val). In some aspects, the added markers are one or more amino acids selected from the group of lysine (Lys), asparagine (Asn), alanine (Ala), aspartic acid (Asp), cysteine (Cys), glutamic acid (Glu), glutamine (Gln), glycine (Gly), isoleucine (Ile), leucine (Leu), methionine (Met), phenylalanine (Phe), proline (Pro), serine (Ser), Threonine (Thr), Tryptophan (Trp), tyrosine (Tyr), and valine (Val). In other aspects, the marker is lysine, histidine, asparagine, or arginine. In some aspects, the marker is selected from the group consisting of lysine (Lys) and asparagine (Asn). In other aspects, the marker is tyrosine. In some aspects, the marker is lysine. In some aspects, the marker is histidine. In other aspects, the marker is asparagine. In other aspects, the marker is arginine. In some aspects the amino acid markers are not fluorescently active. In some aspects, the added markers are vitamins. In some aspects, the markers are one or more vitamins selected form the group comprising cyanocobalamin (B12), folic acid (B9), niacinamide (B3), pyridoxal HCl (B6(AL)), and pyridoxine HCl (B6(INE)). In some aspects, the marker is B12. In some aspects, the marker is B9. In some aspects, the marker is B3. In some aspects, the marker is pyridoxal HCl (B6(AL)). In some aspects, the marker is pyridoxine HCl (B6(INE)).

Cell culture media can comprise materials that exhibit fluorescence, which tend to mask Raman spectroscopy signals. The fluorescence is typically independent of excitation wavelength. When spectra are acquired at two slightly different wavelengths, the fluorescence in each spectrum should be approximately the same, though the Raman peaks should shift with excitation wavelength. Thus, the spectra can be decomposed (e.g., by principal component analysis) to generate a spectrum free of fluorescence.

The present disclosure is also related to a method of monitoring changes in cell culture media that could affect its effectiveness in growing cells in any setting. One aspect of the present disclosure is directed to the measurement of a Raman spectrum of a cell culture media sample to compare it to known spectra of the components of the cell culture media. The methods of the present disclosure are directed to a method for fingerprinting a cell culture media and/or identifying optimal storage conditions for a cell culture media using Raman spectroscopy, comprising: collecting a Raman spectrum of the cell culture media, the cell culture media containing a mixture of one or more components, wherein the Raman spectrum of the cell culture media is compared to one or more reference spectra associated with each of the one or more components or a reference spectra associated with a reference media composition. The methods of the present disclosure are also directed to a method for identifying the rate of change of particular components in a stored cell culture media and/or monitoring the changes of a stored cell culture media comprising: collecting a Raman spectrum of the cell culture media (“collected spectrum”) wherein the Raman spectrum of the cell culture media is compared to a reference spectrum associated with each of the one or more components.

In some aspects, the reference spectrum is to be free of degradation. In some aspects, the collecting a Raman spectrum of the cell culture media is conducted at least about every hour, at least about every two hours, at least about every three hours, at least about every four hours, at least about every five hours, at least every six hours, at least every seven hours, at least about every eight hours, at least about every nine hours, at least about every ten hours, at least about every 11 hours, at least about every 12 hours, at least about every 13 hours, at least about every 14 hours, at least about every 15 hours, at least about every 16 hours, at least about every 17 hours, at least about every 18 hours, at least about every 19 hours, at least about every 20 hours, at least about every 21 hours, at least about every 22 hours, at least about every 23 hours, or at least about every 24 hours. In some aspects, the collecting a Raman spectrum of the cell culture media is conducted at least about every 25 hours, at least about every 26 hours, at least about every 27 hours, at least about every 28 hours, at least about every 29 hours, at least about every 30 hours, at least about every 31 hours, at least about every 32 hours, at least about every 33 hours, at least about every 34 hours, at least about every 35 hours, at least about every 36 hours, at least about every 37 hours, or at least about every 38 hours, at least about every 39 hours, at least about every 40 hours, at least about every 41 hours, at least about every 42 hours, at least about every 43 hours, at least about every 44 hours, at least about every 45 hours, at least about every 46 hours, at least about every 47 hours, at least about every 48 hours, at least about every 49 hours, or at least about every 50 hours. In some aspects, the collecting a Raman spectrum of the cell culture media is conducted about every three hours. In some aspects, the collecting a Raman spectrum of the cell culture media is conducted between about two hours and about three hours, between one hour and four hours, between one hour and three hours, or between two hours and four hours. In some aspects, the collecting a Raman spectrum of the cell culture media is conducted about every three hours.

In some aspects, the Raman spectrum is an average of at least 5 data points, at least 10 data points, at least 15 data points, at least 16 data points, at least 17 data points, at least 18 data points, at least 19 data points, at least 20 data points, at least 21 data points, at least 22 data points, at least 23 data points, at least 24 data points, at least 25 data points, at least 26 data points, at least 27 data points, at least 28 data points, at least 29 data points, at least 30 data points, at least 31 data points, at least 32 data points, at least 33 data points, at least 34 data points, at least 35 data points, at least 36 data points, at least 37 data points, at least 38 data points, at least 39 data points, at least 40 data points, at least 41 data points, at least 42 data points, at least 43 data points, at least 44 data points, at least 45 data points, at least 46 data points, at least 47 data points, at least 48 data points, at least 49 data points, or at least 50 data points. In some aspects, the Raman spectrum is an average of at least 51 data points, at least 52 data points, at least 53 data points, at least 54 data points, at least 55 data points, at least 56 data points, at least 57 data points, at least 58 data points, at least 59 data points, at least 60 data points, at least 61 data points, at least 62 data points, at least 63 data points, at least 64 data points, at least 65 data points, at least 66 data points, at least 67 data points, at least 68 data points, at least 69 data points, at least 70 data points, at least 71 data points, at least 72 data points, at least 73 data points, at least 74 data points, at least 75 data points, at least 76 data points, at least 77 data points, at least 78 data points, at least 79 data points, at least 80 data points, at least 81 data points, at least 82 data points, at least 83 data points, at least 84 data points, at least 85 data points, at least 86 data points, at least 87 data points, at least 88 data points, at least 89 data points, at least 90 data points, at least 91 data points, or at least 92 data points, at least 93 data points, at least 94 data points, at least 95 data points, at least 96 data points, at least 97 data points, at least 98 data points, at least 99 data points, or at least 100 data points. In some aspects, the Raman spectrum for the cell culture media is an average of 35 data points.

In some aspects, each data point is measured every 10 seconds, every 15 seconds, every 20 seconds, every 25 seconds, every 30 seconds, every 35 seconds, every 40 seconds, every 45 seconds, every 50 seconds, every 55 seconds, or every 60 seconds. In some aspects, each data point is measured every 65 seconds, every 70 seconds, every 75 seconds, every 80 seconds, every 85 seconds, every 90 seconds, every 95 seconds, every 100 seconds, every 105 seconds, every 110 seconds, every 115 seconds, or every 120 seconds.

The methods of the present disclosure also involve analysis of the raw Raman spectra collected from the samples. In some aspects, the Raman spectrum is analyzed by a multivariate analysis. In some aspects, the multivariate analysis is a principle component analysis (PCA). In some aspects, the PCA generates a PC score for the Raman spectrum. In some aspects, the multivariate analysis is a partial least squares analysis (PLS) and wherein the analysis produces a calibration prediction model. In some aspects, the method further comprises comparing the collected spectrum of the cell culture media. In some aspects, the comparing comprises a comparison of a PC score of the collected spectrum to a reference PC score of the reference spectrum.

In some aspects, the analysis is based on a predictive model. Established statistical algorithms and methods well-known in the art, useful as models or useful in designing predictive models, can include but are not limited to: Partial Least Squares (PLS) analysis, Standard Normal Variate (SNV) analysis, analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (LogReg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms as are known in the art can be useful in determining subject sub-groups.

Multivariable statistical means, such as principal component analysis (PCA) via intrinsic Raman spectra of the analyte of interest, may be employed. Specifically, linear multivariable models of spectra data sets may be built by establishing principal component vectors (PCs), which will provide the statistically most significant variations in the data sets, and reduce the dimensionality of the sample matrix. This approach involves assigning a score for the PCs of each spectrum collected followed by plotting the spectrum as a single data point in a two-dimensional plot. The plot will reveal clusters of similar spectra, thus individual biological species (analyte and interfering molecules) can be classified and differentiated for even closely related ones.

The methods of the present disclosure require knowledge of one or more reference Raman spectra. The one or more reference spectra may be generated based on the one or more reference samples. Each reference sample may include one or more basic (biochemical) components. A processor may be configured to generate one or more reconstructed Raman images based on the one or more Raman spectra. The processor may be configured to remove fluorescence background based on the reference Raman spectra.

In some aspects, the method further comprises determining that the cell culture media is degraded when the PC score of the collected spectrum is different from the reference PC score of the reference spectrum at least by about 10, at least by about 11, at least by about 12, at least by about 13, at least by about 14, at least by about 15, at least by about 16, at least by about 17, at least by about 18, at least by about 19, at least by about 20, at least by about 21, at least by about 22, at least by about 23, at least by about 24, at least by about 25, at least by about 26, at least by about 27, at least by about 28, at least by about 29, or at least by about 30. In some aspects, the method further comprises determining that the cell culture media is degraded when the PC score of the collected spectrum is higher than the reference PC score of the reference spectrum. In some aspects, the method further comprises determining that the cell culture media is degraded when the PC score of the collected spectrum is lower than the reference PC score of the reference spectrum.

In some aspects, the cell culture media is not degraded and the non-degraded cell culture media improves the viability of the cells in culture as compared to a degraded media by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 21%, by about 22%, by about 23%, by about 24%, by about 25%, by about 26%, by about 27%, by about 28%, by about 29%, by about 30%.

In some aspects, the cell culture media is not degraded and the non-degraded cell culture media improves the viable cell density of the cells in culture as compared to a degraded media by about 1×10⁶ cells/mL, by about 2×10⁶ cells/mL, by about 3×10⁶ cells/mL, by about 4×10⁶ cells/mL, by about 5×10⁶ cells/mL, by about 6×10⁶ cells/mL, by about 7×10⁶ cells/mL, by about 8×10⁶ cells/mL, by about 9×10⁶ cells/mL, by about 10×10⁶ cells/mL, by about 11×10⁶ cells/mL, by about 12×10⁶ cells/mL, by about 13×10⁶ cells/mL, by about 14×10⁶ cells/mL, by about 15×10⁶ cells/mL, by about 16×10⁶ cells/mL, by about 17×10⁶ cells/mL, by about 18×10⁶ cells/mL, by about 19×10⁶ cells/mL, by about 20×10⁶ cells/mL, by about 21×10⁶ cells/mL, by about 22×10⁶ cells/mL, by about 23×10⁶ cells/mL, by about 24×10⁶ cells/mL, by about 25×10⁶ cells/mL, by about 26×10⁶ cells/mL, by about 27×10⁶ cells/mL, by about 28×10⁶ cells/mL, by about 29×10⁶ cells/mL, or by about 30×10⁶ cells/mL.

In some aspects, the cell culture media is not degraded and the non-degraded cell culture media improves an antibody production titer of the cells in culture as compared to a degraded media by about 0.1 g/L, by about 0.2 g/L, by about 0.3 g/L, by about 0.4 g/L, by about 0.5 g/L, by about 0.6 g/L, by about 0.7 g/L, by about 0.8 g/L, by about 0.9 g/L, by about 1.0 g/L, by about 1.1 g/L, by about 1.2 g/L, by about 1.3 g/L, by about 1.4 g/L, by about 1.5 g/L, by about 1.6 g/L, by about 1.7 g/L, by about 1.8 g/L, by about 1.9 g/L, by about 2.0 g/L, by about 2.1 g/L, by about 2.2 g/L, by about 2.3 g/L, by about 2.4 g/L, by about 2.5 g/L, by about 2.6 g/L, by about 2.7 g/L, by about 2.8 g/L, by about 2.9 g/L, by about 3.0 g/L, by about 3.1 g/L, by about 3.2 g/L, by about 3.3 g/L, by about 3.4 g/L, by about 3.5 g/L, by about 3.6 g/L, by about 3.7 g/L, by about 3.8 g/L, by about 3.9 g/L, by about 4.0 g/L, by about 4.1 g/L, by about 4.2 g/L, by about 4.3 g/L, by about 4.4 g/L, by about 4.5 g/L, by about 4.6 g/L, by about 4.7 g/L, by about 4.8 g/L, by about 4.9 g/L, by about 5.0 g/L, by about 5.1 g/L, by about 5.2 g/L, by about 5.3 g/L, by about 5.4 g/L, by about 5.5 g/L, by about 5.6 g/L, by about 5.7 g/L, by about 5.8 g/L, by about 5.9 g/L, or by about 6.0 g/L.

In some aspects, a marker with a known reference spectrum can be analyzed via the methods of the present disclosure is present in a cell culture composition. The methods of the present disclosure can also be useful to detect degradation of a composition via the addition of a known component as a marker. In some aspects, a marker is added to the cell culture media. In some aspects, the marker is selected from the group consisting of lysine, histidine, asparagine, alanine, aspartic acid, cysteine, glutamine, glutamic acid, glycine isoleucine, leucine, lysine, methionine, phenylalanine, proline, serine, threonine, tryptophan, valine, and arginine. In some aspects, the marker is tyrosine. In other aspects, the marker is a sugar. In some aspects, the marker is selected from the group consisting of fructose, maltose, hexose, arabinose, or sucrose. In some aspects, the marker is not glucose. In some aspects, the marker is not lactate. In some aspects, the marker is a vitamin. In some aspects, the vitamin is selected from the group consisting of Vitamin A, Vitamin C, Vitamin D, Vitamin E, Vitamin K, Vitamin B6, Vitamin B12, folate, thiamine, riboflavin, niacin, pantothenic acid, biotin, and folate. In some aspects, the marker is a vitamin is selected from the group consisting of cyanocobalamin (B12), folic acid (B9), niacinamide (B3), pyridoxal HCl (B6(AL)), and pyridoxine HCl (B6(INE)).

In some aspects, the Raman spectrum is measured in the range of from about 500 cm⁻¹ to about 1700 cm⁻¹, from about 500 cm⁻¹ to about 1800 cm⁻¹, from about 500 cm⁻¹ to about 1900 cm⁻¹, from about 500 cm⁻¹ to about 2000 cm⁻¹, from about 500 cm⁻¹ to about 2100 cm⁻¹, from about 500 cm⁻¹ to about 2200 cm⁻¹, from about 500 cm⁻¹ to about 2300 cm⁻¹, from about 500 cm⁻¹ to about 2400 cm⁻¹, from about 500 cm⁻¹ to about 2500 cm⁻¹, from about 500 cm⁻¹ to about 2600 cm⁻¹, from about 500 cm⁻¹ to about 2700 cm⁻¹, from about 500 cm⁻¹ to about 2800 cm⁻¹, from about 500 cm⁻¹ to about 2900 cm⁻¹, or from about 500 cm⁻¹ to about 3000 cm⁻¹. In some aspects, the Raman spectrum is measured in the range of 500 cm⁻¹ to 3000 cm⁻¹.

In some aspects, the Raman spectrum is measured in the range of from about 500 cm⁻¹ to about 3000 cm⁻¹, from about 600 cm⁻¹ to about 3000 cm⁻¹, from about 600 cm⁻¹ to about 2900 cm⁻¹, from about 700 cm⁻¹ to about 2900 cm⁻¹, from about 700 cm⁻¹ to about 2800 cm⁻¹, from about 800 cm⁻¹ to about 2800 cm⁻¹, from about 800 cm⁻¹ to about 2700 cm⁻¹, from about 900 cm⁻¹ to about 2700 cm⁻¹, from about 900 cm⁻¹ to about 2600 cm⁻¹, from about 1000 cm⁻¹ to about 2600 cm⁻¹, from about 1000 cm⁻¹ to about 2500 cm⁻¹, from about 1100 cm⁻¹ to about 2500 cm⁻¹, from about 1100 cm⁻¹ to about 2400 cm⁻¹, or from about 1200 cm⁻¹ to about 2400 cm⁻¹, from about 1200 cm⁻¹ to about 2300 cm⁻¹, from about 1300 cm⁻¹ to about 2300 cm⁻¹, from about 1300 cm⁻¹ to about 2200 cm⁻¹, from about 1400 cm⁻¹ to about 2200 cm⁻¹, from about 1400 cm⁻¹ to about 2100 cm⁻¹, from about 1500 cm⁻¹ to about 2100 cm⁻¹, from about 1500 cm⁻¹ to about 2000 cm⁻¹, from about 1600 cm⁻¹ to about 2000 cm⁻¹, from about 1600 cm⁻¹ to about 1900 cm⁻¹, from about 1700 cm⁻¹ to about 1900 cm⁻¹, or from about 1800 cm⁻¹ to about 1900 cm⁻¹.

In some aspects, the method further comprises determining that the cell culture media is stable when the PC score of the collected spectrum is the same as or similar to the reference PC score of the reference spectrum by about 20 or less, by about 19 or less, by about 18 or less, by about 17 or less, by about 16 or less, by about 15 or less, by about 14 or less, by about 13 or less, by about 12 or less, by about 11 or less, by about 10 or less, by about 9 or less, by about 8 or less, by about 7 or less, by about 6 or less, by about 5 or less, by about 4 or less, by about 3 or less, by about 2 or less, or by about 1 or less.

In some aspects, the cell culture media is determined for storage for about eight days, for about nine days, for about ten days, for about 11 days, for about 12 days, about 15 day, for about 16 days, for about 17 days, for about 18 days, for about 19 days, for about 20 days, for about 21 days, for about 22 days, for about 23 days, for about 24 days, for about 25 days, for about 26 days, for about 27 days, for about 28 days, for about 29 days, for about 30 days, for about a month, for about 1.5 months, for about 40 days, for about 50 days, for about 60 days, for about two months, for about 70 days, for about 80 days, for about 90 days, for about three months, for about 100 days, for about 110 days, for about 120 days, for about 4 months, for about five months, or for about six months. In some aspects, the cell culture media is determined for storage for about 12 months, for about 18 months, for about 24 months, for about 30 months, for about 36 months, for about 42 months, for about 48 months, for about 54 months, or for about 60 months.

The methods of the present disclosure further comprises monitoring cell culture during a manufacturing process. In some aspects, the method further comprises monitoring an upstream cell culture process. In some aspects, the upstream cell culture process comprises a batch reactor process. In some aspects, the upstream cell culture process comprises a perfusion reactor process. In other aspects, the upstream cell culture process comprises a fed batch reactor process.

In mammalian cell culture, the cell culture media can comprise up to about 100 compounds and more. For example, carbohydrates (e.g. for generation of energy by catabolic reactions or as building blocks by anaplerotic reactions), amino acids (e.g. building blocks for cellular protein and product in case of therapeutic protein production), lipids and/or fatty acids (e.g. for cellular membrane synthesis), DNA and RNA (e.g. for growth and cellular mitosis and meiosis), vitamins (e.g. as co-factors for enzymatic reactions), trace elements, different salts, growth factors, carriers, transporters, etc. These components or compound groups are required to fulfill the complex nutritional requirements of mammalian cells in a technical cultivation environment. In some aspects, the culture media used for the present disclosure comprises classical cell culture media, e.g., DMEM (Dulbecco's Modified Eagle's Medium) where all components and all concentrations are published. Development of such cell culture media go back to the late 1950s and are comprehensively described in the academic literature. Another example is Ham's F12 (Ham's Nutrient Mixture F12) that was developed in the 1970s, or mixtures/modifications of such classical cell culture such as DMEM:F12 (Dulbecco's Modified Eagle's Medium/Ham's Nutrient Mixture F12) that were developed in the 1970s and 1980s. Another culture medium useful for the present disclosure comprises RPMI. RPMI was developed in the 1970s by Moore et al. at the Roswell Park Memorial Institute (hence the acronym RPMI). Different variants are used in animal cell culture, for example RPMI-1640. Although many of these classical media were developed decades ago, these formulations still form the basis for much of the cell culture research occurring today and represent state of the art in animal cell culture for media with completely known composition and completely known concentrations for each compound. All of these media are commercially available and can be obtained from suppliers (e.g. from Sigma-Aldrich.

In other aspects, the cell culture media useful for the present disclosure includes commercial cell culture media, for example, a commercially available medium ActiCHO (by PAA) consisting of a basal medium (ActiCHO P) and a feed medium (ActiCHO Feed A+B), which is chemically defined according to supplier definition (only single chemicals, free of animal derived substances, growth factors, peptides, and peptones). The two feeds consist of concentrated amino acids, vitamins, salts trace elements and carbon source (Feed A) and selected amino acids in concentrated form (Feed B). In some aspects, the culture media comprises Ex-Cell CD CHO (SAFC Biosciences). This medium is animal component free, chemically defined according to SAFC, serum-free, and formulation is also proprietary. In other aspects, the culture media comprises CD CHO (Life technologies). This medium is protein free, serum-free, and chemically defined according to Life technologies. It does not contain proteins/peptides of animal, plant or synthetic origin or undefined lysates/hydrolysates. This CD CHO basal medium can be combined with feed media named Efficient Feed A, B, and C. The feeds are animal origin-free and the components are contained in higher concentrations. The feeds are chemically defined. No proteins, no lipids, no growth factors, no hydrolysates and no components of unknown composition are used. It contains a carbon source, concentrated amino acids, vitamins and trace elements. Another feed that is commercially available can be obtained by Thermo Fisher, named Cell Boost 1-6. It is chemically defined according to Thermo Fisher, protein free, and animal derived components free. Cell Boost 1 and 2 contain amino acids, vitamins, and glucose. Cell Boost 3 contains amino acids, vitamins, glucose, and trace elements. Cell Boost 4 contains amino acids, vitamins, glucose, trace elements, and growth factors. And Cell Boost 5 and 6 contain amino acids, vitamins, glucose, trace elements, growth factors, lipids, and cholesterol. Amino acids

Amino acids have an essential role for protein synthesis, both for cellular protein and for the production of the product in case of recombinant proteins or protein-derived substances. For examples, proteins are synthesized by the cellular machinery from single amino acids molecules to form larger proteins or protein complexes. In mammalian cell cultivation the essential amino acids need to be provided with the cell culture medium, since mammalian cells are not able to synthesize essential amino acids from other precursors and building blocks. Amino acids are also biochemically important because these molecules have two functional groups (amino group and an acidic group) which enables them to interact with other biological molecules. For these reasons cell culture media containing amino acids are often also supplemented with a variety of (defined and undefined) small peptides, hydrolysates, proteins and protein mixtures from different origins (animal derived, plant derived or chemically defined). Therefore, one or more amino acids can be used as a marker for Raman spectroscopy according to the present disclosure.

One or more amino acids useful for the present disclosure includes the twenty natural amino acids that are encoded by the universal genetic code, typically the L-form (i.e., L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine). In other aspects, the one or more amino acids for the present disclosure include non-naturally occurring amino acids. The amino acids (e.g., glutamine and/or tyrosine) can be provided as dipeptides with increased stability and/or solubility, for example, containing an L-alanine (L-ala-x) or L-glycine extension (L-gly-x), such as glycyl-glutamine and alanyl-glutamine. Further, cysteine can also be provided as L-cystine. The term “amino acids” as used herein encompasses all different salts thereof, such as (without being limited thereto) L-arginine monohydrochloride, L-asparagine monohydrate, L-cysteine hydrochloride monohydrate, L-cystine dihydrochloride, L-histidine monohydrochloride dihydrate, L-lysine monohydrochloride and hydroxyl L-proline, L-tyrosine disodium dehydrate. The exact form of the amino acids is not of importance for this disclosure, unless characteristics such as solubility, osmolarity, stability, purity are impaired. In some aspects, L-arginine is used as L-arginine×HCl, L-asparagine is used as L-asparagine×H20, L-cysteine is used as L-cysteine×HCl×H20, L-cystine is used as L-cystine×2 HCl, L-histidine is used as L-histidine×HCl×H20 and L-tyrosine is used as L-tyrosine×2 Na×2 H20, wherein each amino acid form can be selected independent of the other or together or any combination thereof. Also encompassed are dipeptides comprising one or two of the relevant amino acids. For example, L-glutamine is often added in the form of dipeptides, such as L-alanyl-L-glutamine to the cell culture medium for improved stability and reduced ammonium built up in storage or during long-term culture.

In some aspects, the present methods are used to produce one or more polypeptides. In some aspects, the polypeptides comprise an antibody or antigen binding portion thereof. In other aspects, the polypeptide includes fusion proteins consisting of an immunoglobulin component (e.g. the Fc component) and a growth factor (e.g. an interleukin), antibodies or any antibody derived molecule formats or antibody fragments.

In other aspects, the polypeptide comprises naturally occurring proteins. In other aspects, the polypeptide includes proteins, polypeptides, fragments thereof, peptides, fusion proteins all of which can be expressed in the selected host cell, e.g., a recombinant protein, i.e., a protein encoded by a recombinant DNA resulting from molecular cloning. Such polypeptides can be antibodies, enzymes, cytokines, lymphokines, adhesion molecules, receptors and derivatives or fragments thereof, and any other polypeptides that can serve as agonists or antagonists and/or have therapeutic or diagnostic use or can be used as research reagent. In some aspects, the polypeptide is a secreted protein or protein fragment, e.g., an antibody or antibody fragment or an Fc-fusion protein.

In some aspects, the polypeptides are produced from cultured cells. In some aspects, the cells are prokaryotes. In bacterial systems, a number of expression vectors can be advantageously selected depending upon the use intended for the protein molecule being expressed. For example, when a large quantity of such a protein is to be produced, for the generation of pharmaceutical compositions of a protein molecule, vectors which direct the expression of high levels of protein products that are readily purified can be desirable.

In other aspects, the cells are eukaryotes. In some aspects, the cells are mammalian cells. In some aspects, the cells are selected from Chinese hamster ovary (CHO) cells, HEK293 cells, mouse myeloma (NSO), baby hamster kidney cells (BHK), monkey kidney fibroblast cells (COS-7), Madin-Darby bovine kidney cells (MDBK), and any combination thereof. In one aspect, the cells are Chinese hamster ovary cells. In some aspects, the cells are insect cells, e.g., Spodoptera frugiperda cells.

In other aspects, the cells are mammalian cells. Such mammalian cells include but are not limited to CHO, VERO, BHK, Hela, MDCK, HEK 293, NIH 3T3, W138, BT483, Hs578T, HTB2, BT2O and T47D, NSO, CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

In some aspects, the mammalian cells are CHO cells. In some aspects the CHO cell is CHO-DG44, CHOZN, CHO/dhfr−, CHOK1SV GS-KO, or CHO-S. In some aspects, the CHO cell is CHO-DG4. In some aspects, the CHO cell is CHOZN.

Other suitable CHO cell lines disclosed herein include CHO-K (e.g., CHO K1), CHO pro3−, CHO P12, CHO-K1/SF, DUXB11, CHO DUKX; PA-DUKX; CHO pro5; DUK-BII or derivatives thereof.

In some aspects, the proteins produced by the culture media according to the present disclosure are fusion proteins. A “fusion” or “fusion” protein comprises a first amino acid sequence linked in frame to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some aspects, the proteins produced by the culture media according to the present disclosure are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain aspects, antibodies described herein are IgG antibodies, or a class (e.g., human IgG1 or IgG4) or subclass thereof. In a specific aspect, the antibody is a humanized monoclonal antibody. In another specific aspect, the antibody is a human monoclonal antibody, preferably that is an immunoglobulin. In certain aspects, an antibody described herein is an IgG1, or IgG4 antibody.

In some aspects, the protein described herein is an “antigen-binding domain,” “antigen-binding region,” “antigen-binding fragment,” and similar terms, which refer to a portion of an antibody molecule which comprises the amino acid residues that confer on the antibody molecule its specificity for the antigen (e.g., the complementarity determining regions (CDR)). The antigen-binding region can be derived from any animal species, such as rodents (e.g., mouse, rat or hamster) and humans.

In some aspects, the protein is an anti-LAG3 antibody, an anti-CTLA-4 antibody, an anti-TIM3 antibody, an anti-NKG2a antibody, an anti-ICOS antibody, an anti-CD137 antibody, an anti-KIR antibody, an anti-TGFβ antibody, an anti-IL-10 antibody, an anti-B7-H4 antibody, an anti-Fas ligand antibody, an anti-mesothelin antibody, an anti-CD27 antibody, an anti-GITR antibody, an anti-CXCR4 antibody, an anti-CD73 antibody, an anti-TIGIT antibody, an anti-OX40 antibody, an anti-PD-1 antibody, an anti-PD-L1 antibody, an anti-IL8 antibody, or any combination thereof. In some aspects, the protein is Abatacept NGP. In other aspects, the protein is Belatacept NGP.

In some aspects, the protein is an anti-GITR (glucocorticoid-induced tumor necrosis factor receptor family-related gene) antibody. In some aspects, the anti-GITR antibody has the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841, WO2016/057846, WO 2018/013818, or other anti-GITR antibody described or referred to herein, all of which are incorporated herein in their entireties.

In other aspects, the protein is an anti-LAG3 antibody. Lymphocyte-activation gene 3, also known as LAG-3, is a protein which in humans is encoded by the LAG3 gene. LAG3, which was discovered in 1990 and is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right. Examples of anti-LAG3 antibodies include, but are not limited to, the antibodies in WO 2017/087901 A2, WO 2016/028672 A1, WO 2017/106129 A1, WO 2017/198741 A1, US 2017/0097333 A1, US 2017/0290914 A1, and US 2017/0267759 A1, all of which are incorporated herein in their entireties.

In some aspects, the protein is an anti-CXCR4 antibody. CXCR4 is a 7 transmembrane protein, coupled to Gl. CXCR4 is widely expressed on cells of hemopoietic origin, and is a major co-receptor with CD4+ for human immunodeficiency virus 1 (HIV-1) See Feng, Y., Broeder, C. C., Kennedy, P. E., and Berger, E. A. (1996) Science 272, 872-877. Examples of anti-CXCR4 antibodies include, but are not limited to, the antibodies in WO 2009/140124 A1, US 2014/0286936 A1, WO 2010/125162 A1, WO 2012/047339 A2, WO 2013/013025 A2, WO 2015/069874 A1, WO 2008/142303 A2, WO 2011/121040 A1, WO 2011/154580 A1, WO 2013/071068 A2, and WO 2012/175576 A1, all of which are incorporated herein in their entireties.

In some aspects, the protein is an anti-CD73 (ecto-5′-nucleotidase) antibody. In some aspects, the anti-CD73 antibody inhibits the formation of adenosine. Degradation of AMP into adenosine results in the generation of an immunosuppressed and pro-angiogenic niche within the tumor microenvironment that promotes the onset and progression of cancer. Examples of anti-CD73 antibodies include, but are not limited to, the antibodies in WO 2017/100670 A1, WO 2018/013611 A1, WO 2017/152085 A1, and WO 2016/075176 A1, all of which are incorporated herein in their entireties.

In some aspects, the protein is an anti-TIGIT (T cell Immunoreceptor with Ig and ITIM domains) antibody. TIGIT is a member of the PVR (poliovirus receptor) family of immunoglobin proteins. TIGIT is expressed on several classes of T cells including follicular B helper T cells (TFH). The protein has been shown to bind PVR with high affinity; this binding is thought to assist interactions between TFH and dendritic cells to regulate T cell dependent B cell responses. Examples of anti-TIGIT antibodies include, but are not limited to, the antibodies in WO 2016/028656 A1, WO 2017/030823 A2, WO 2017/053748 A2, WO 2018/033798 A1, WO 2017/059095 A1, and WO 2016/011264 A1, all of which are incorporated herein by their entireties.

In some aspects, the protein is an anti-OX40 (i.e., CD134) antibody. OX40 is a cytokine of the tumor necrosis factor (TNF) ligand family. OX40 functions in T cell antigen-presenting cell (APC) interactions and mediates adhesion of activated T cells to endothelial cells. Examples of anti-OX40 antibodies include, but are not limited to, WO 2018/031490 A2, WO 2015/153513 A1, WO 2017/021912 A1, WO 2017/050729 A1, WO 2017/096182 A1, WO 2017/134292 A1, WO 2013/038191 A2, WO 2017/096281 A1, WO 2013/028231 A1, WO 2016/057667 A1, WO 2014/148895 A1, WO 2016/200836 A1, WO 2016/100929 A1, WO 2015/153514 A1, WO 2016/002820 A1, and WO 2016/200835 A1, all of which are incorporated herein by their entireties.

In some aspects, the protein is an anti-IL8 antibody. IL-8 is a chemotactic factor that attracts neutrophils, basophils, and T-cells, but not monocytes. It is also involved in neutrophil activation. It is released from several cell types in response to an inflammatory stimulus.

In some aspects, the protein is Abatacept (marketed as ORENCIA®). Abatacept (also abbreviated herein as Aba) is a drug used to treat autoimmune diseases like rheumatoid arthritis, by interfering with the immune activity of T cells. Abatacept is a fusion protein composed of the Fc region of the immunoglobulin IgG1 fused to the extracellular domain of CTLA-4. In order for a T cell to be activated and produce an immune response, an antigen presenting cell must present two signals to the T cell. One of those signals is the major histocompatibility complex (MHC), combined with the antigen, and the other signal is the CD80 or CD86 molecule (also known as B7-1 and B7-2).

In some aspects, the protein is Belatacept (trade name NULOJIX®). Belatacept is a fusion protein composed of the Fc fragment of a human IgG1 immunoglobulin linked to the extracellular domain of CTLA-4, which is a molecule crucial in the regulation of T cell costimulation, selectively blocking the process of T-cell activation. It is intended to provide extended graft and transplant survival while limiting the toxicity generated by standard immune suppressing regimens, such as calcineurin inhibitors. It differs from abatacept (ORENCIA®) by only 2 amino acids.

The present disclosure is also related to an apparatus for carrying out measurements of Raman spectra using an apparatus capable of analyzing cell culture media. In some aspects, the apparatus comprises a mixture of one or more components in the cell culture media, the apparatus comprising: a cell culture media sample holder for holding the cell culture media sample; a laser source for illuminating the cell culture media sample held by the cell culture media sample holder; a particle motion detector positioned to detect motion of one or more components in the cell culture media in the cell culture media sample held by the cell culture media sample holder; and a spectral detector positioned to receive a spectrum from the cell culture media sample resulting from illumination by the laser source.

Various aspects of the disclosure are described in further detail in the following subsections. The present disclosure is further illustrated by the following examples which should not be construed as further limiting. The contents of all references cited throughout this application are expressly incorporated herein by reference.

### EXAMPLES

The capability of a specific Raman technique to “fingerprint” media composition and the rate-of-change of such composition was evaluated in a series of studies. The results show that this technique can detect errors due to media preparation, monitor changes in media as it ages, and detect variations due to different modes of degradation (light versus heat). Media subjected to degradation conditions were also tested in a production bioreactor run to provide functional confirmation of the Raman-detected degradation. Raman models were built to quantitatively monitor specific amino acids and vitamins within the media, and the accuracy of the models was evaluated against known concentrations. Based on the below examples, Raman can be utilized as an analytical tool to monitor media changes and that Raman based models can predict the level of each component within the media.

### Example 1

### Apparatus Setup

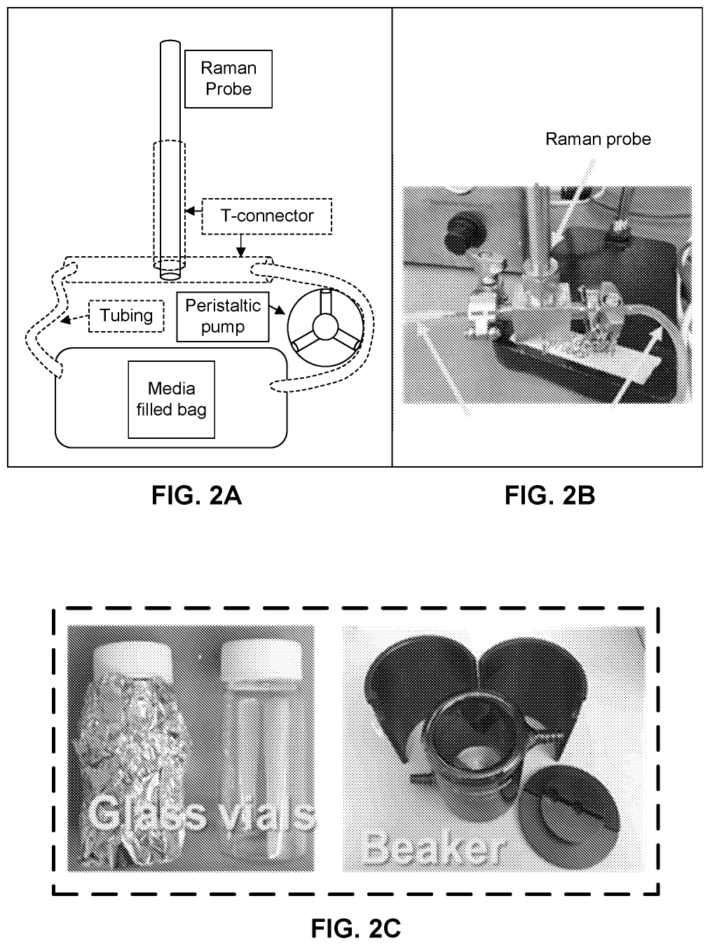

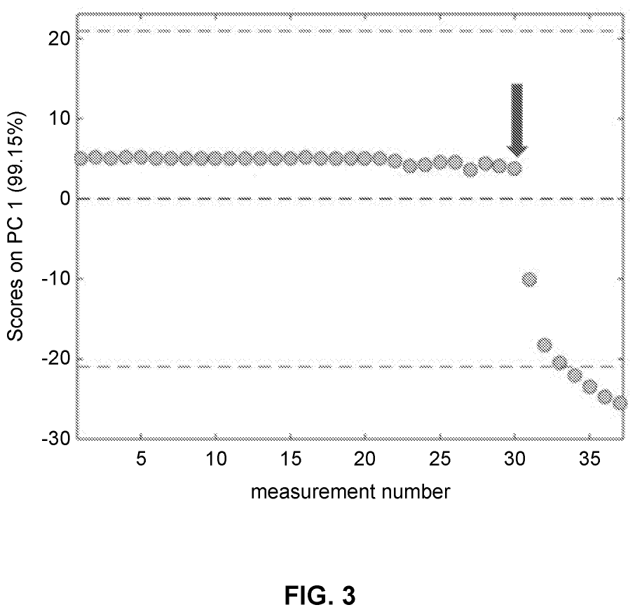

Raman is able to provide measurements from samples provided online or offline. For online measurements, we used a stainless steel T connector (FIGS. 2A and 2B). In order to prevent contamination, first the T-connector setup, with attached tubing (on the bottom side) and Raman probe (on the top end) was autoclaved and then connected to the bag following sterile tube connection procedure. The media was run through the connector using a peristaltic pump and data collected at adjustable time increments. During offline measurements, either a 20 mL glass vial or a 200 mL beaker was used to collect media. Both containers were covered to eliminate light interference with Raman scattering (FIG. 3). Both feed and basal media were evaluated. Basal media is the media in the process at the time of inoculation. Feed media has higher concentrations of many components and is added to the culture throughout the production process.

Exposure conditions were optimized to yield averaged Raman results from 35 data points which were collected over 12 minutes (one data point every 20 seconds). The averaged data provided optimal resolution within a reasonable amount of time and without saturating the signal. The Raman spectra of each measurement were analyzed by multivariate analysis.

Multivariate analysis is a technique used to interpret many variables simultaneously. Principle component analysis (PCA) is a multivariate-based technique that can help narrow down a large set of variables to a smaller set containing the needed information. In PCA, orthogonal variables termed Principal Components (PC) are generated in the same data space occupied by the raw Raman spectra, forming linear combinations of the original variables (i.e. wavenumbers). The first PC value accounts for as much of the variability in the data as possible, and each succeeding value accounts for as much of the remaining variability as possible. The PC scores can show which spectra are similar or different, i.e., if the Raman spectra of two measurements are alike the PC scores will group together, if not, the measurements will diverge. PC loading plots reveal contributions of each wavenumber to a particular PC and tell where the major difference(s) come from. All calculations were performed using PLS Toolbox supplemented by Matlab (v. 8.6.2).

The raw Raman data were first preprocessed before doing PCA in order to reduce or eliminate irrelevant and systematic variations in the data. The preprocessing is mainly needed to offset baseline and remove background noise, i.e., normalizing the data to eliminate potential scaling or gain effects and variable centering. In this study the optimum PCA results were obtained using the following preprocessing steps: taking the first-order derivative of the spectra using the Savitzky-Golay algorithm to remove the baseline, using second order polynomials fitted with filter width of 15; normalizing the spectra using Standard Normal Variate (SNV); and mean centering the data to compare the difference to the entire original data matrix.

### Example 2

### Real-Time Monitoring of Changes in the Media Due to Precipitation

The outlined Raman technique was utilized to monitor feed media in real-time, using the apparatus described above and shown in FIG. 2. The setup was as described above. Raman measurements were obtained using the approach described above every 3 hours and the changes in the media over a four day period were monitored by applying PCA to raw Raman spectra as described above.

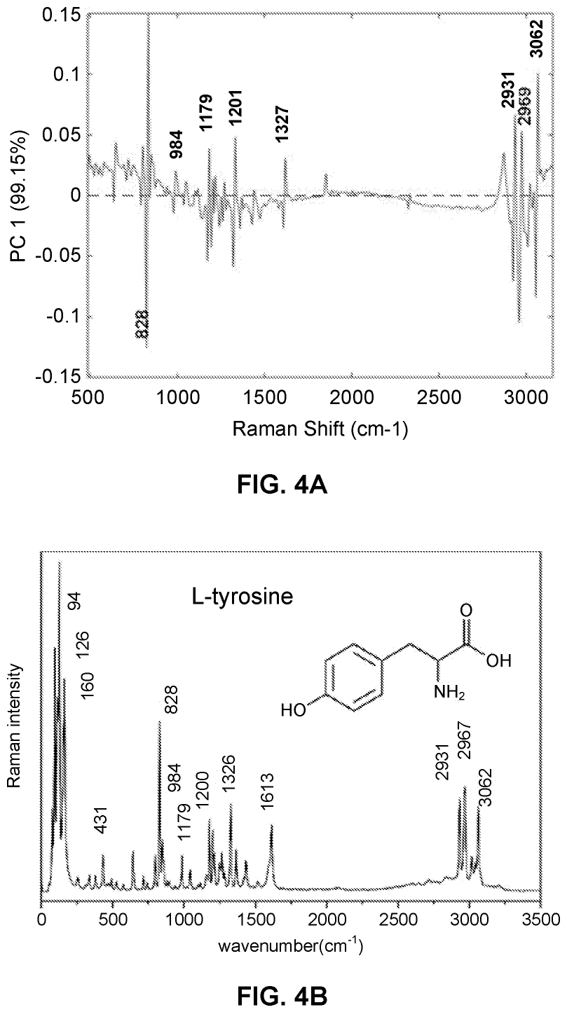

In this experiment, the region selected for analysis was 500-3000 cm⁻¹ since this is the range most pertinent to evaluating changes in feed media components. The examination of PCs, as determined from percent variance plots, was used to investigate changes in the spectral features of Raman data. The data showed that >99% of all the spectral variation could be accounted for by PC1. The first principal component, PC1, with an explained spectral variance of 99.15%, is shown in FIG. 3 where the changes during each measurement were captured. The PC1 scores are similar up to the 30th measurements, around 90 hours (pointed by arrow) meaning the sample was unchanged for the first 90 hours, and after that the media started to physically change at this time point resulted in deviation of the PC1 scores. To better understand the source of the chemical change, the PC1 loading values were plotted against the Raman shift (FIG. 4). The PC1 loading plot reveals at which wavenumber the major changes occur which is where there are X-intercepts with sharp change in the PC1 scores (y-axis). The corresponding wavenumbers are labeled in FIGS. 4A and 4B. From previous experience, it was hypothesized that the change was due to L-tyrosine precipitation. FIG. 4B shows the major Raman shift for tyrosine crystals as occurring at 825, 984, 1179, 1200, 1326, 1613, 2931, 2967, and 3062 cm⁻¹, which matched precisely with the Raman shifts observed on the PC1 loading plot (FIG. 4A). Thus, this Raman technique was able to correctly identify the source of media change through a precise and unequivocal identification of tyrosine. We would anticipate that any amino acid precipitated out of media could be monitored with similar efficiency by this technique. The capability of this Raman technique to detect media preparation errors was examined. Sodium phosphate is an essential component of growth media, however, if too much or too little is added the resulting media can be detrimental to cell growth. Thus, it is crucial to have the ability to confirm the phosphate concentration prior to use. In this example, the amount of sodium phosphate in the media was measured at the normal level of 1.5 g/L and compared to twice the normal level (3 g/L).

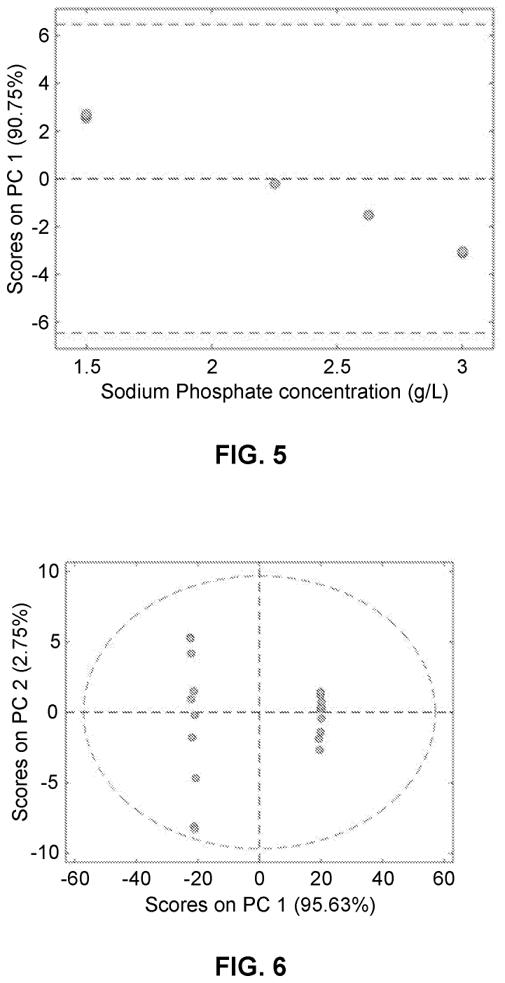

Media including 1.5 g/L sodium phosphate, media including 3 g/L sodium phosphate, and mixtures of these two solutions to yield g/L concentrations of 1.5, 2.25, 2.6 and 3, were monitored by this Raman technique and the data evaluated using the PCA method. Sodium phosphate has a major Raman shift at ˜1000 cm⁻¹ and so the region selected for analysis was 700-1200 cm⁻¹. The data showed that >93% of all the spectral variation was captured by the first two PCs. As shown in FIG. 5, the first principal component, PC1, was able to accurately measure the relative amounts of phosphate added to the two media. Specifically, the levels of sodium phosphate were shown to be indirectly correlated with the PC1 scores. The 1.5 and 3.0 g/L samples were run in triplicate to show that the technique was reproducible. Importantly, the data showed that this technique was able to differentiate sodium phosphate level changes to as low as 0.4 g/L given an appropriate standard to measure against. Considering that each Raman measurement took approximately 12 minutes, the effort required to discover the error would be far more advantageous from a time and cost perspective than discovering the error upon batch failure.

### Example 3

### Monitoring Media Stability

Media expiry is a critical process parameter reaching optimal growth rates and achieving expected product attributes which is typically determined in time and resource intensive functional testing. The storage conditions of basal and feed media are 2-8° C. and room temperature, respectively, protected from light. To examine the ability of the technique to identify stability markers, the Raman data collected from basal and feed media stored under standard conditions were compared to media exposed to light (10 KLux) or heat (36° C.) for two and four weeks. Freshly-prepared and aged (3 months at standard storage conditions) media were also compared to see if this Raman technique could identify media differences under less extreme circumstances.

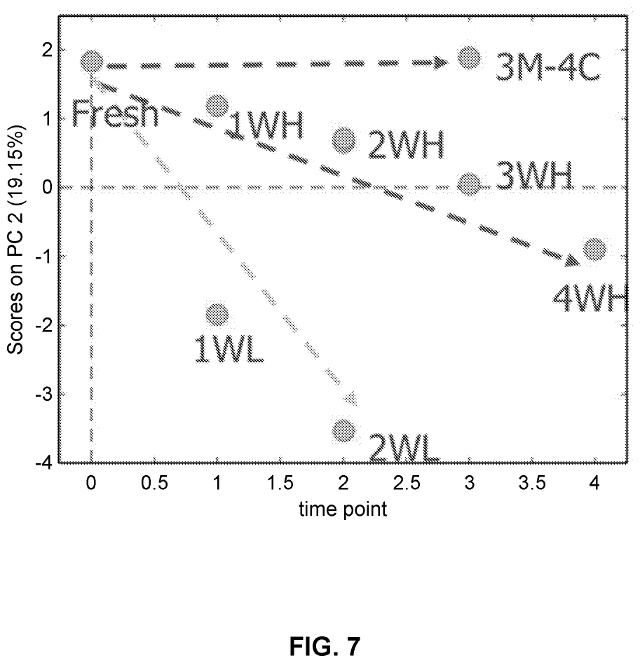

All media samples were monitored with Raman spectroscopy and evaluated using PCA as described previously. In this evaluation, the data was grouped first by PC scores to differentiate basal from feed media then evaluated by PCA to further parse the more subtle changes. The first two PC scores represent the vast majority of the changes. FIG. 6 shows the PC1-PC2 plane where PC1 captures ˜96% and PC2 captures ˜3% of the major changes. PC1 separates basal from feed media, with basal media having positive scores and feed media having negative scores (FIG. 6). Within each PC1 cluster the samples were differentiated by their degradation mode and the extent of degradation along the PC2 axis. Samples from basal and feed media were further processed separately with PCA. For basal media, the data showed that two PC scores accounted for >80% of all the spectral variation, thus these two scores were used going forward. The PC2 score accounting for ˜19% of the representative variance, was plotted against media degradation time under various conditions (4, 36° C., light-exposed, etc.) in FIG. 7. In this analysis, high heat causes more change to the basal media than light for the similar time points. Data also suggests that storage at standard conditions of 2-8° C. for up to 3 months does not cause a change.

To correlate the Raman analysis with impacts on process performance, fresh and degraded basal media was used in production runs to see if the PC trends observed by Raman could predict performance.

The production setup was as follows: 15 mL AMBR mini bioreactors were used to grow a CHO cell line producing a monoclonal antibody in a 14 day production run. To evaluate the effect of basal, differently degraded basal were used for cell production and the cells were fed with fresh feed once a day. The AMBR system was connected to a Vi-CELL Cell Viability Analyzer from Beckman Coulter which monitored cell viability and viable cell density (VCD) over the AMBR production run. Antibody titer was measured with the CEDEX HT from Roche. The % viability, VCD and titer are the three main characteristics of the cell production evaluation.

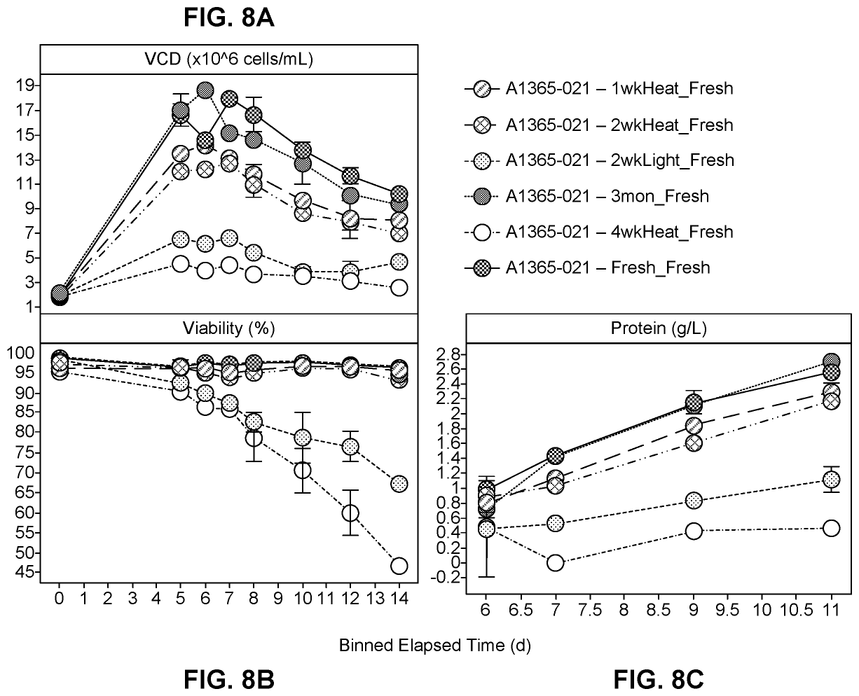

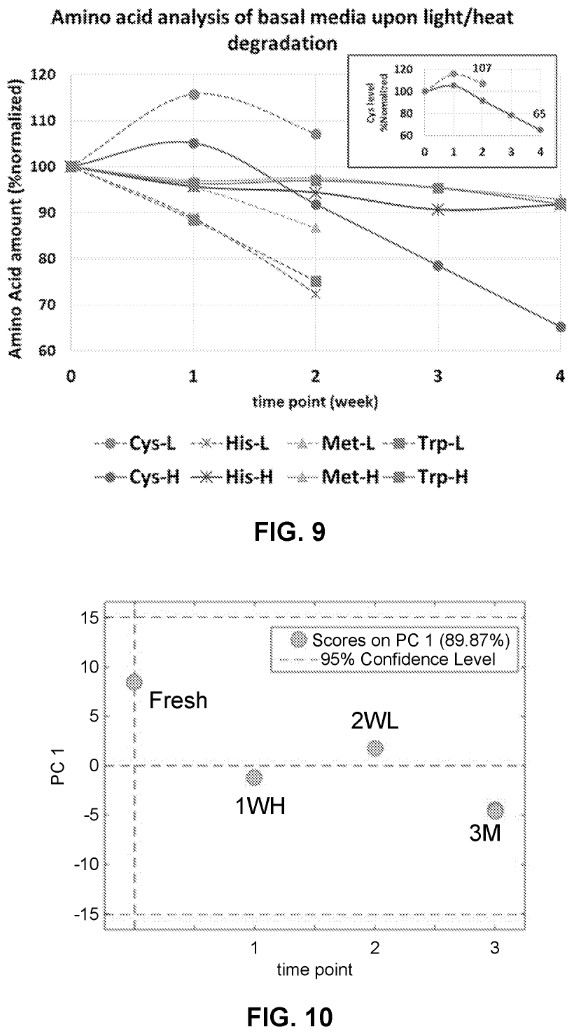

Cell growth parameter results from the Ambr15 basal degradation study are presented in FIGS. 8A-8C. Cell viability and density in both fresh and 3 month aged basal stored at 2-8° C. were very similar, but large differences were observed from fresh media control when using basal media degraded by heat or light. Similar results were observed when evaluating titers, although 4 weeks of heat yielded a worsening effect on cell growth than 2 weeks light-exposure, which was the opposite of what we would expect when comparing to the Raman data. This discrepancy can be explained by noting that light and heat degrade by differing mechanisms, thus will not necessarily degrade the same media components. Raman captures the changes in the sample regardless of what the component is, whereas production results show the effect of degraded components on cells. This data suggests that cells were more affected by component(s) degraded by heat rather than light.

To further understand these changes, amino acid content of each degraded media was measured using Reverse Phase HPLC technique. The results showed that while Cys, Met, Trp and His were the media-containing amino acids most affected by degradation, Met, Trp and His were mostly degraded by 2 weeks of light degradation and Cys was unchanged due to light effect. On the other hand Cys was the only amino acid that was degraded due to heat and other amino acids were not affected significantly. FIGS. 8A-8C represent the changes in the levels of these four amino acids in basal media during the light or heat exposure over 2 and 4 weeks respectively. Cysteine form can impact media pH and it is a required media component. So, while Cys degradation due to heat affected cell growth and production profiles observed in FIGS. 8A-8C, Met, Trp and His on the other hand did not affect cell growth significantly.

The impact of basal media degradation to protein quality was also assessed by iCIEF. The iCIEF results showed increased basic charge variants with degradation of Met, Trp and His upon light degradation which was not the case for other conditions. Raman spectroscopy was able to detect media degradation important to both process performance and product quality.

The changes in the feed media were processed separately by PCA as well. Greater than 95% of all the spectral variation in the feed data was accounted for with two PCs. The PC1 score plot in FIG. 10 presents 1 week heat degradation, 2 weeks light degradation and 3 months aged feed samples at RT (typical storage limits for feed are about 3 weeks). The results showed that as feed media degraded the scores moved toward the negative side of the PC1 axis. Comparing the PC1 score changes from the fresh sample showed that more degradation was observed during the 3 months at RT compared to the stressed conditions. 2 weeks of light exposure had the least degradation of the group.

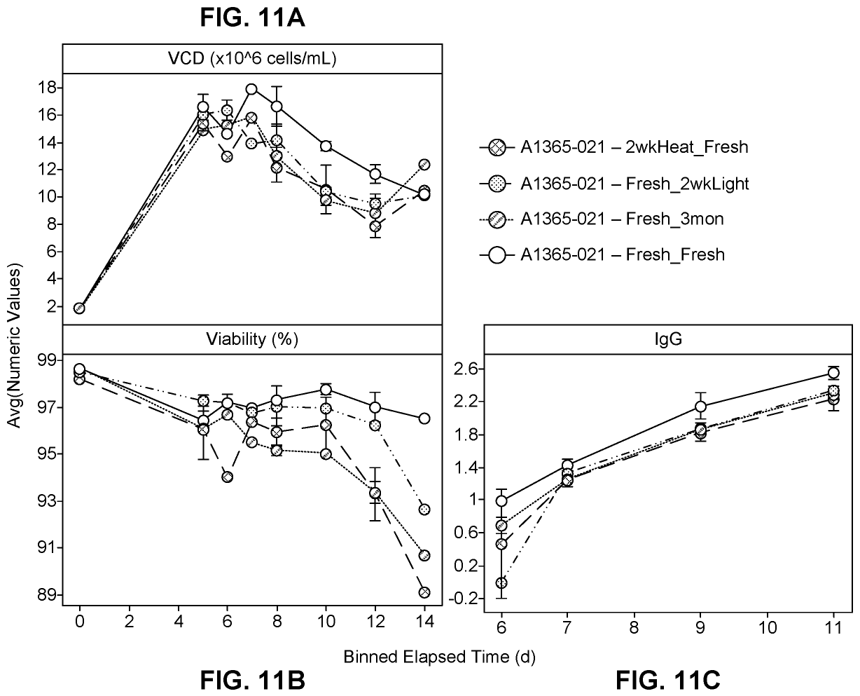

The same samples were chosen for a production run using 15 mL AMBR bioreactors using fresh basal media and fresh and aged feeds for the 14 day run. The three major production results are presented in FIGS. 11A-11C. The VCD and IgG results showed higher peak VCD and higher IgG concentration when fresh feed was used, and the degraded feed under light, RT or heat resulted in similar VCD but did not impact the IgG titer. The % viability stayed above 96% when fresh feed was used during the entire 14 days of production, however, when degraded feed was used the viability began dropping at day 10. The 3 months aged feed and 1 week heat had the lowest viability of ˜89%, and the 2 week light-exposed feed had viability at ˜92%. These results were in agreement with the changes observed by Raman and subsequent PCA based data evaluation.

### Example 4

### Developing Models for Quantitative Analysis of Media Components

In previous sections the capability of Raman to monitor changes in media either due to media preparation errors or degradation were discussed. The following studies were devoted to expanding the quantitative applications of this Raman technique to identify the rate of change of particular components of the media.

Four amino acids, Lysine (Lys), histidine (His), asparagine (Asn) and arginine (Arg), were selected for this study as they are easily dissolved in media. Note that Lys, Asn, and Arg are not fluorescently active thus can only be detected directly by a technique like Raman, versus other methods like Excitation Emission Spectroscopy. Vitamins evaluated included cyanocobalamin (B12), folic acid (B9), niacinamide (B3), pyridoxal HCl (B6(AL)), and pyridoxine HCl (B6(INE)).

The Raman spectra of each measurement were processed by Partial Least Squares (PLS) analysis. PLS identifies only the factors, i.e. “latent variables” or LV, accounting for the variance in the input data relevant to making a prediction. This is in contrast to PCA, where the PCs are selected solely based on the relative amount of variation they represent.

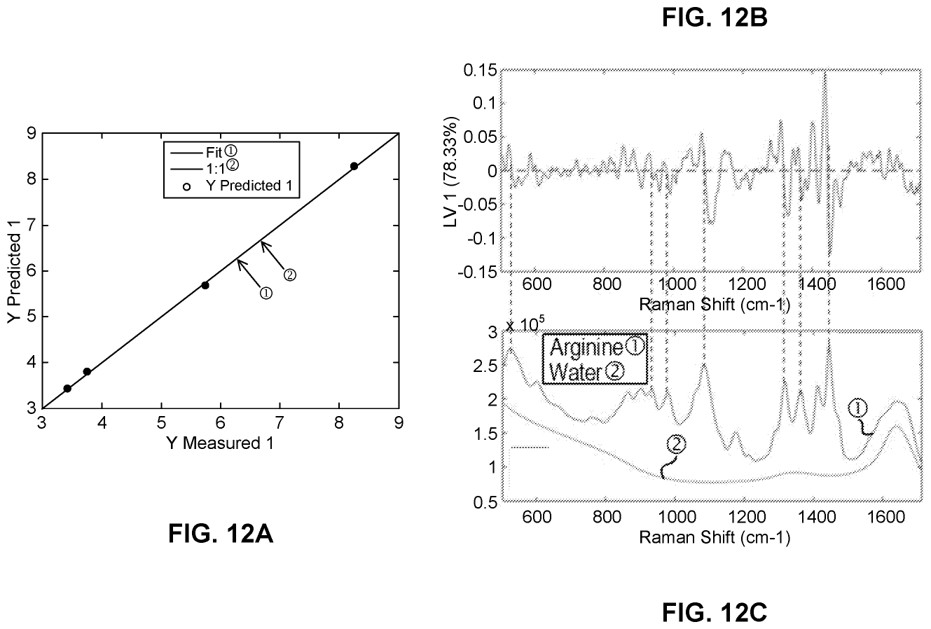

In each experiment, different concentrations of each of the above chemicals were spiked into the feed media then evaluated by Raman. The data was then processed by PLS to build a calibration prediction model. For example, FIG. 12A shows the prediction calibration model for arginine based on LV1 using PLS analysis, and FIG. 12B reveals the wavenumbers correlated to the major changes relevant to make the prediction model. FIG. 12C shows the Raman spectra of Arginine solution, the dashed arrows lining up the major Raman shift specific to Arginine (FIG. 12C) with the wavelengths which the model was built upon (FIG. 12B), confirming the model was build based on the changes in Arginine level. Unique profiles can be provided in this fashion for each chemical and the same approach was taken to build a prediction model for each of the four amino acids and four vitamins in this study.

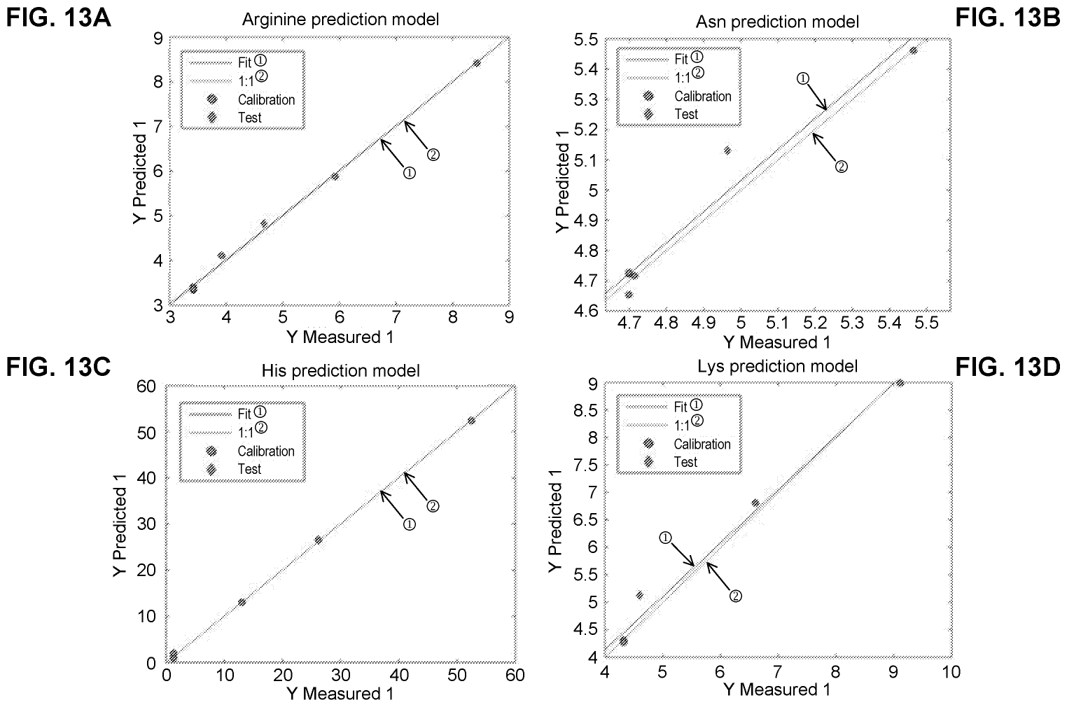

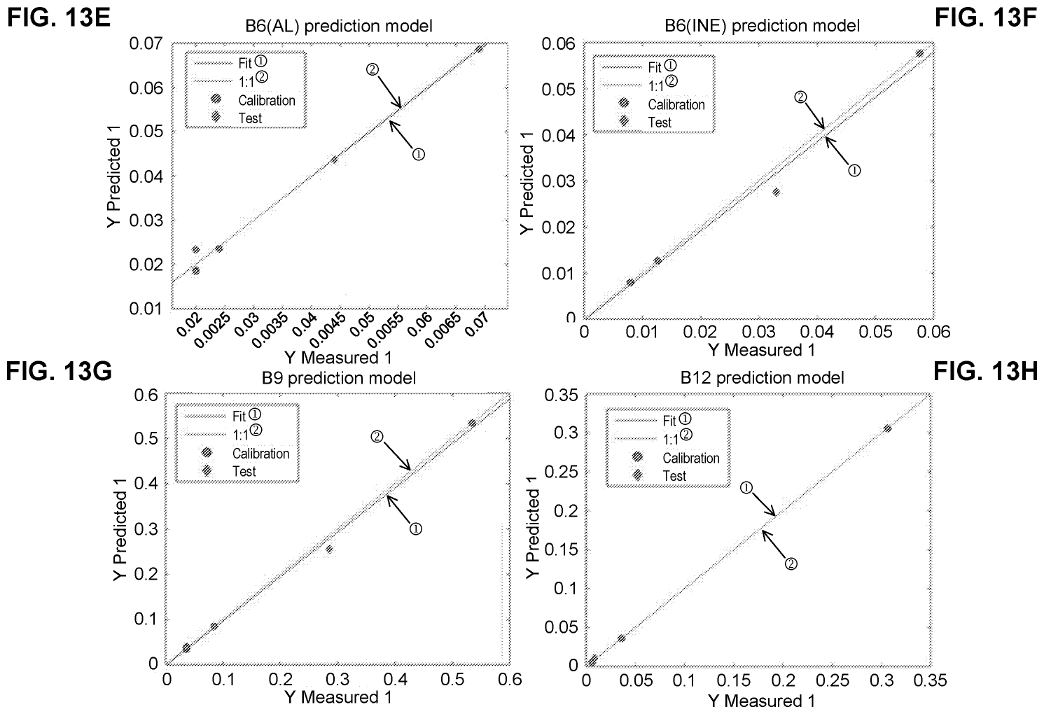

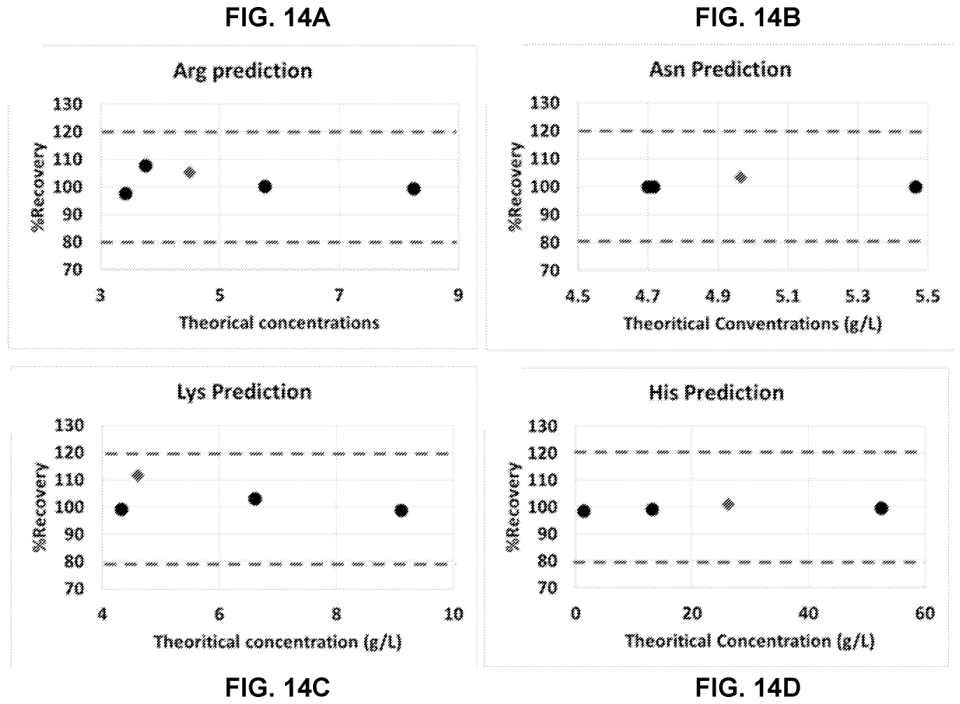

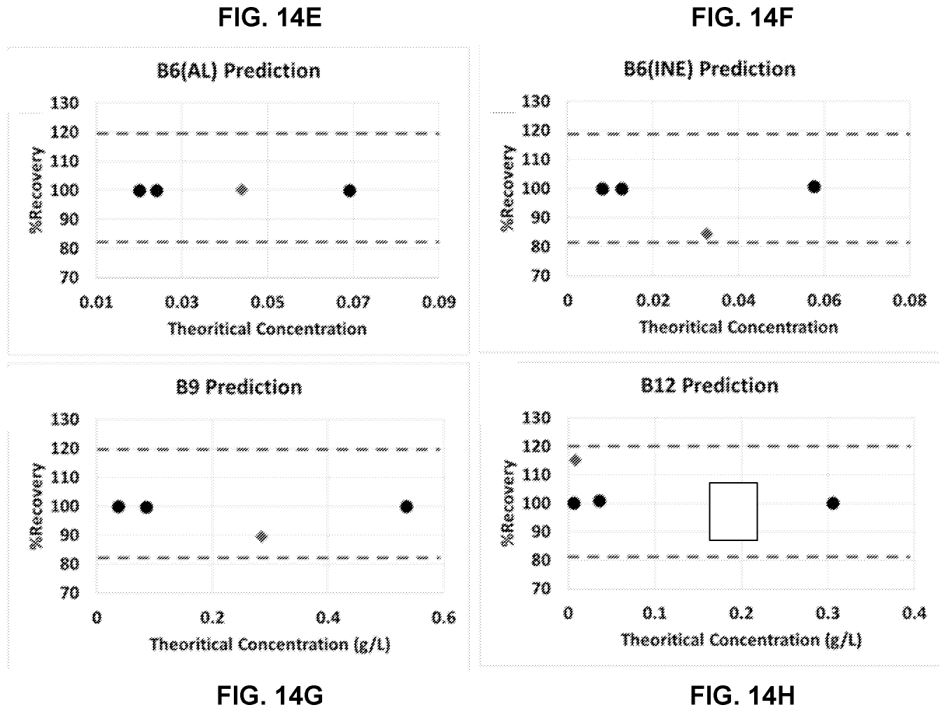

To test the accuracy of each prediction model, the models were first used to predict a known concentration of the related chemical. The predicted level was then compared to the theoretical level to evaluate the accuracy of the model for each media component tested. FIGS. 13A-13H show prediction models for each tested media component, where the fit line is the fitted line of the calibration curve built using known concentrations of each chemical. The dots show the concentration levels used to build the prediction model. The 1:1 line shows a 1:1 ratio between measured and predicted values, so the stronger the prediction model the better fit and 1:1 line overlay. The diamonds in FIGS. 13A-13H show the predicted level of the unknown sample for each of the amino acids and vitamins in this study. As another way to represent the accuracy of the experimental result compared to theoretical, FIGS. 14A-14H show % recovery for each chemical. Here, the circles represent the points used to build the calibration curve and the diamond is the predicted concentration using the calibration curve. The % recoveries were calculated using Equation 1:

$$\% recovery = \frac{Predicted value}{theoretical value} \times 100 \qquad Equation 1$$

The dashed lines show the 80% to 120% recovery boundaries. Overall, the experimental values matched closely to the theoretical values, with typical recoveries of 90-110% of theoretical.

### Example 5

### Generation of Reference Spectra

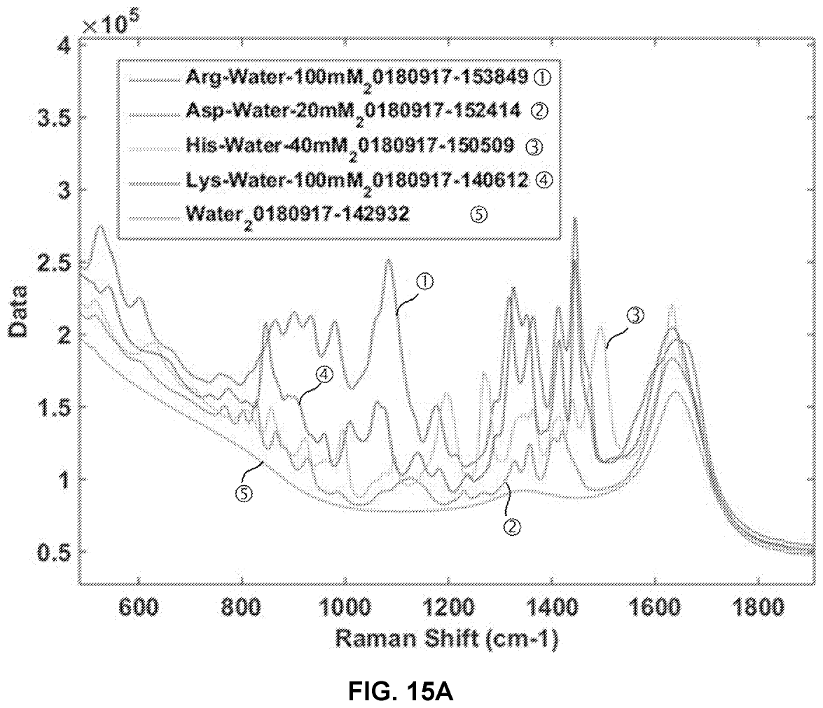

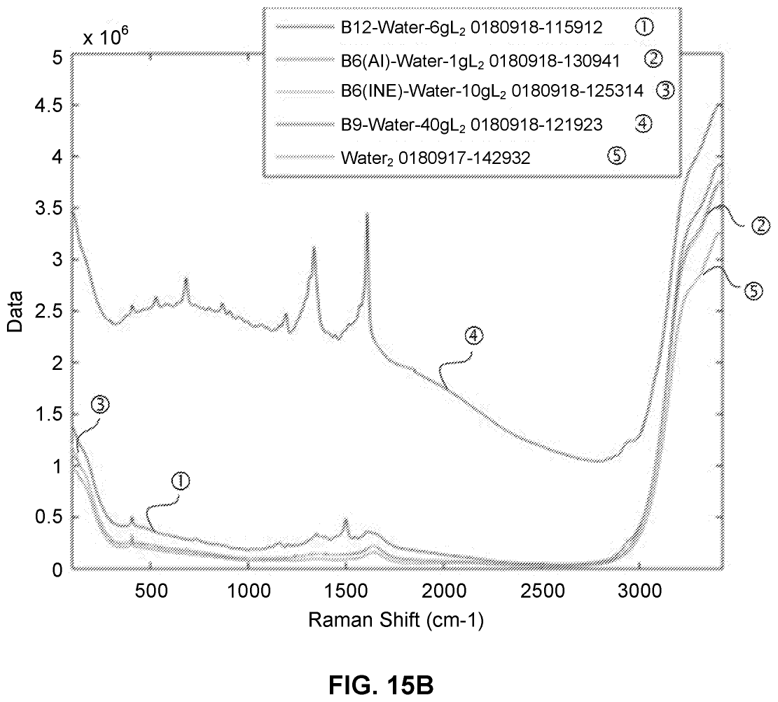

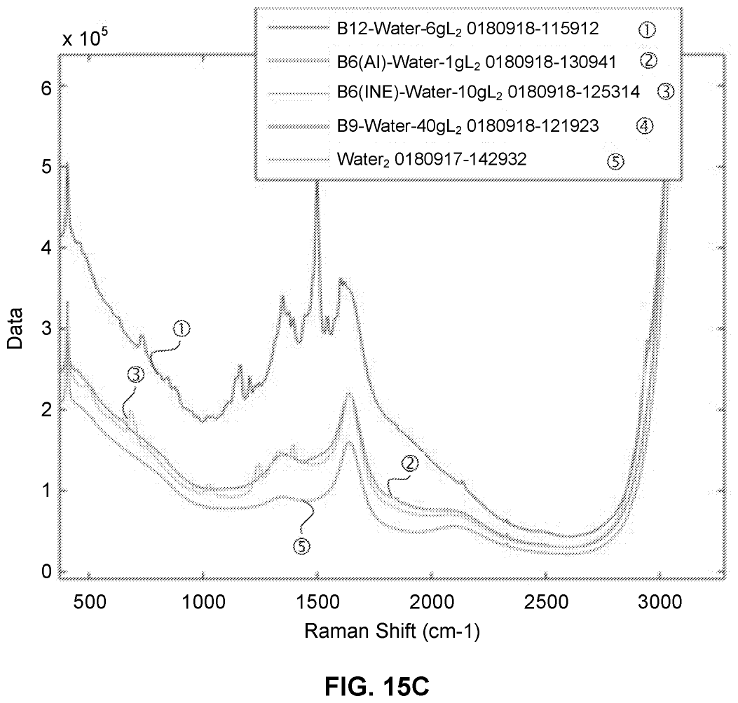

Generation of reference spectra for amino acids and vitamins useful for Raman analysis were carried out, and can be seen in FIGS. 15A-15C. The three factors that affect the optimum exposure conditions for Raman spectrum measurements are resolution, signal saturation, and a reasonable spectra collection time. The resolution can be improved by increasing the number of acquisitions and the exposure length per acquisition, however increasing these values will increase the measurement period and can cause signal saturation. Three conditions were evaluated initially, the first condition consisted of co-addition of 35 spectral acquisition at 20 sec each for total of approximately 11 min, the second condition consisted of co-addition of 35 spectral acquisition at 40 sec each for total of approximately 22 min, and the third condition consisted of co-addition of 10 spectral acquisition at 75 sec each for total of approximately 12 min. The third condition resulted in over exposure in several cases, and the second condition did not result in any improvement in signal resolution compared to the first. Ultimately, the first condition was selected as the method for Raman spectra collection.

1. A method for fingerprinting a cell culture media and/or identifying optimal storage conditions for a cell culture media using Raman spectroscopy, comprising: collecting a Raman spectrum of the cell culture media, the cell culture media containing a mixture of one or more components, wherein the Raman spectrum of the cell culture media is compared to one or more reference spectra associated with each of the one or more components or a reference spectra associated with a reference media composition.

2. A method for identifying the rate of change of particular components in cell culture media and/or monitoring the changes of a cell culture media in real-time using Raman spectroscopy, comprising: collecting a Raman spectrum of the cell culture media (“collected spectrum”), wherein the Raman spectrum of the cell culture media is compared to a reference spectrum associated with each of the one or more components.

3. The method of claim 1 or 2, wherein the reference spectrum is to be free of degradation.

4. The method of any one of claims 1 to 3, wherein the collecting a Raman spectrum of the cell culture media is conducted about every hour, about every two hours, about every three hours, about every four hours, about every five hours, about every six hours, about every seven hours, about every eight hours, about every nine hours, about every ten hours, about every 11 hours, about every 12 hours, about every 13 hours, about every 14 hours, about every 15 hours, about every 16 hours, about every 17 hours, about every 18 hours, about every 19 hours, about every 20 hours, about every 21 hours, about every 22 hours, about every 23 hours, or about every 24 hours.

5. The method of claim 4, wherein the Raman spectrum is an average of at least 5 data points, at least 10 data points, at least 15 data points, at least 16 data points, at least 17 data points, at least 18 data points, at least 19 data points, at least 20 data points, at least 21 data points, at least 22 data points, at least 23 data points, at least 24 data points, at least 25 data points, at least 26 data points, at least 27 data points, at least 28 data points, at least 29 data points, at least 30 data points, at least 31 data points, at least 32 data points, at least 33 data points, at least 34 data points, at least 35 data points, at least 36 data points, at least 37 data points, at least 38 data points, at least 39 data points, at least 40 data points, at least 41 data points, at least 42 data points, at least 43 data points, at least 44 data points, at least 45 data points, at least 46 data points, at least 47 data points, at least 48 data points, at least 49 data points, or at least 50 data points.

6. The method of claim 5, wherein each data point is measured every 10 seconds, every 15 seconds, every 20 seconds, every 25 seconds, every 30 seconds, every 35 seconds, every 40 seconds, every 45 seconds, every 50 seconds, every 55 seconds, or every 60 seconds.

7. The method of any one of claims 1 to 6, wherein the Raman spectrum is analyzed by a multivariate analysis.

8. The method of claim 7, wherein the multivariate analysis is a principle component analysis (PCA).

9. The method of claim 7, wherein the PCA generates a PC score for the Raman spectrum.

10. The method of claim 7, wherein the multivariate analysis is a partial least squares analysis (PLS) and wherein the analysis produces a calibration prediction model.

11. The method of any one of claims 1 to 10, further comprising comparing the collected spectrum of the cell culture media.

12. The method of claim 11, wherein the comparing comprises a comparison of a PC score of the collected spectrum to a reference PC score of the reference spectrum.

13. The method of claim 11 or 12, further comprising determining that the cell culture media is degraded when the PC score of the collected spectrum is different from the reference PC score of the reference spectrum.

14. The method of claim 11 or 12, further comprising determining that the cell culture media is degraded when the PC score of the collected spectrum is higher than the reference PC score of the reference spectrum.

15. The method of claim 11 or 12, further comprising determining that the cell culture media is degraded when the PC score of the collected spectrum is lower than the reference PC score of the reference spectrum.

16. The method of any one of claims 13-15, wherein the cell culture media is degraded and the degraded cell culture media reduces the viability of a plurality of cells in culture as compared to a non-degraded media by about 1%, by about 2%, by about 3%, by about 4%, by about 5%, by about 6%, by about 7%, by about 8%, by about 9%, or by about 10%.

17. The method of any one of claims 13-16, wherein the cell culture media is degraded and the degraded cell culture media reduces the viable cell density of the cells in culture as compared to a non-degraded media by about 1×10⁶ cells/mL, by about 2×10⁶ cells/mL, by about 3×10⁶ cells/mL, by about 4×10⁶ cells/mL, by about 5×10⁶ cells/mL, by about 6×10⁶ cells/mL, by about 7×10⁶ cells/mL, by about 8×10⁶ cells/mL, by about 9×10⁶ cells/mL, by about 10×10⁶ cells/mL, by about 11×10⁶ cells/mL, or by about 12×10⁶ cells/mL.

18. The method of any one of claims 13-17 wherein the cell culture media is degraded and the degraded cell culture media reduces an antibody production titer of the cells in culture as compared to a non-degraded media by about 0.1 g/L, by about 0.2 g/L, by about 0.3 g/L, by about 0.4 g/L, by about 0.5 g/L, by about 0.6 g/L, by about 0.7 g/L, by about 0.8 g/L, by about 0.9 g/L, by about 1.0 g/L, by about 1.1 g/L, by about 1.2 g/L, by about 1.3 g/L, by about 1.4 g/L, by about 1.5 g/L, by about 1.6 g/L, by about 1.7 g/L, by about 1.8 g/L, by about 1.9 g/L, by about 2.0 g/L, by about 2.1 g/L, by about 2.2 g/L, by about 2.3 g/L, by about 2.4 g/L, by about 2.5 g/L, by about 2.6 g/L, by about 2.7 g/L, by about 2.8 g/L, by about 2.9 g/L, or by about 3.0 g/L.

19. The method of any one of claims 1-18, wherein a marker for the one or more reference spectra is added to the cell culture media.

20. The method of claim 19, wherein the marker is not glucose.

21. The method of claim 20, wherein the marker is not lactate.

22. The method of any one of claims 19-21, wherein the marker is an amino acid or a vitamin.

23. The method of claim 22, wherein the marker is selected from the group consisting of lysine (Lys), asparagine (Asn), alanine (Ala), aspartic acid (Asp), cysteine (Cys), glutamic acid (Glu), glutamine (Gln), glycine (Gly), isoleucine (Ile), leucine (Leu), methionine (Met), phenylalanine (Phe), proline (Pro), serine (Ser), Threonine (Thr), Tryptophan (Trp), tyrosine (Tyr), or valine (Val).

24. The method of claim 23, wherein the marker is tyrosine.

25. The method of any one of claims 19-21, wherein the marker is selected from the group consisting of cyanocobalamin (B12), folic acid (B9), niacinamide (B3), pyridoxal HCl (B6(AL)), and pyridoxine HCl (B6(INE)).

26. The method of any one of claims 1-25, wherein the Raman spectrum is measured in the range of from about 500 cm⁻¹ to about 1700 cm⁻¹, from about 500 cm⁻¹ to about 1800 cm⁻¹, from about 500 cm⁻¹ to about 1900 cm⁻¹, from about 500 cm⁻¹ to about 2000 cm⁻¹, from about 500 cm⁻¹ to about 2100 cm⁻¹, from about 500 cm⁻¹ to about 2200 cm⁻¹, from about 500 cm⁻¹ to about 2300 cm⁻¹, from about 500 cm⁻¹ to about 2400 cm⁻¹, from about 500 cm⁻¹ to about 2500 cm⁻¹, from about 500 cm⁻¹ to about 2600 cm⁻¹, from about 500 cm⁻¹ to about 2700 cm⁻¹, from about 500 cm⁻¹ to about 2800 cm⁻¹, from about 500 cm⁻¹ to about 2900 cm⁻¹, or from about 500 cm⁻¹ to about 3000 cm⁻¹.

27. The method of any one of claim 26, wherein the Raman spectrum is measured in the range of 500 cm⁻¹ to 3000 cm⁻¹.

28. The method of any one of claims 1-27, comprising determining that the cell culture media is stable when the PC score of the collected spectrum is the same as or similar to the reference PC score of the reference spectrum by about 10 or less, by about 9 or less, by about 8 or less, by about 7 or less, by about 6 or less, by about 5 or less, by about 4 or less, by about 3 or less, by about 2 or less, or by about 1 or less.

29. The method of claim 1-28, wherein the cell culture media is determined for storage for about eight days, for about nine days, for about ten days, for about 11 days, for about 12 days, about 15 day, for about 16 days, for about 17 days, for about 18 days, for about 19 days, for about 20 days, for about 21 days, for about 22 days, for about 23 days, for about 24 days, for about 25 days, for about 26 days, for about 27 days, for about 28 days, for about 29 days, for about 30 days, for about a month, for about 1.5 months, for about 40 days, for about 50 days, for about 60 days, for about two months, for about 70 days, for about 80 days, for about 90 days, for about three months, for about 100 days, for about 110 days, for about 120 days, for about 4 months, for about five months, or for about six months.

30. An apparatus for spectroscopic investigation of a cell culture media sample comprising a mixture of one or more components in the cell culture media, the apparatus comprising: a cell culture media sample holder for holding the cell culture media sample; a laser source for illuminating the cell culture media sample held by the cell culture media sample holder; a particle motion detector positioned to detect motion of one or more components in the cell culture media in the cell culture media sample held by the cell culture media sample holder; and a spectral detector positioned to receive a spectrum from the cell culture media sample resulting from illumination by the laser source.
